(19) United States
(12) Patent Application Publication
(10) Pub. No.: US 2019/0308956 A1
(43) Pub. Date: Oct. 10, 2019

(54) PIPERAZINE DERIVATIVES AS ANTIVIRAL AGENTS WITH INCREASED THERAPEUTIC ACTIVITY

(71) Applicants: SERVICIO ANDALUZ DE SALUD, Sevilla (ES); UNIVERSIDAD DE SEVILLA, Sevilla (ES)

(72) Inventors: Javier SÁNCHEZ CÉSPEDES, Sevilla (ES); María Eugenia PACHÓN IBÁÑEZ, Sevilla (ES); Jerónimo PACHÓN DÍAZ, Sevilla (ES); Pablo MARTÍNEZ AGUADO, Sevilla (ES); Tania CEBRERO CANGUEIRO, Sevilla (ES); José Manuel VEGA PÉREZ, Sevilla (ES); Fernando IGLESIAS GUERRA, Sevilla (ES); Margarita VEGA HOLM, Sevilla (ES); José Ignacio CANDELA LENA, Sevilla (ES); Sarah MAZZOTTA, Sevilla (ES)

(21) Appl. No.: 16/079,473

(22) PCT Filed: Feb. 23, 2017
(86) PCT No.: PCT/EP2017/054252

§ 371 Date: Aug. 23, 2018

(30) Foreign Application Priority Data
Feb. 23, 2016 (EP) 16382073.1

(51) Int. Cl. C07D 405/06; C07D 295/195; C07D 295/21; A61P 31/20; A61P 31/22

(52) U.S. Cl. CPC C07D 405/06; C07D 295/195; A61P 31/22; A61P 31/20; C07D 295/21

(57) ABSTRACT

The present invention provides 2-phenylpiperazine derivatives having a benzofuran-2-yl group which contributes to increase the antiviral activity as well as, for some substituents, the CC₅₀, giving more active and less cytotoxic compounds. Although further optimization and characterization of their mechanisms of action will be required for these compounds, they represent strong hit candidates for the development of a new class of antiviral compounds.

### FIELD OF THE INVENTION

The present invention can be included in the field of medicine, in particular in the field of antiviral and antibacterial agents.

### BACKGROUND OF THE INVENTION

Human adenoviruses (HAdVs) are non-enveloped, double stranded DNA viruses consisting of more than 60 serotypes divided into 7 subgroups or species (A-G). In healthy individuals these viruses are responsible for diseases ranging from acute respiratory and ocular infections to more severe enteric diseases, but are rarely associated with severe clinical symptoms.

On the other hand, the improvement of the immunosuppressive therapies together with the progress of viral diagnostic tools has revealed HAdV to be one of the more common causes of potentially lifethreatening viral diseases associated with transplantation and a leading cause of increased infections in pediatric units. In pediatric allogenic hematopoetic stem cell transplant (HSCT) recipients HAdV infections occur in frequencies between 3-47% with associated mortality rates of 2-80%. Moreover, in solid organ transplant (SOT) recipients HAdV infections occur in approximately 10% of liver and heart transplant recipients, and in 22% of lung recipients.

Despite this significant clinical impact, nowadays there are no approved antiviral therapies for HAdV infections. Non-specific therapeutic options to treat HAdV infections in immunosuppressed patients include the use of broadly acting antivirals such as ganciclovir, acyclovir, vidarabine, ribavirin and cidofovir, with highly variable results. Ribavirin and cidofovir are the most frequently used, however, neither has been approved for specific use in HAdV infections. Ribavirin has variable activity against different HAdV types, displaying maximum activity against subgroup C; however, the plasma concentrations reached by ribavirin are 10 times below the required IC50 value.

On the other hand, cidofovir exhibits antiviral activity against all HAdV species but has low oral bioavailability, significant toxicity (tubular necrosis), and does not confer long term protection. Moreover, the company Gilead Sciences, the manufacturer of cidofovir (Vistide®has formally requested the annulment of the Authorization for the Vistide® commercialization in Europe due to problems with its manufacture and the availability of other therapeutic options for the indication it was approved for (retinitis by cytomegalovirus). While a lipidic conjugate of cidofovir, CMX001, is currently being tested in a Phase II clinical trial other potential antiviral agents with increased therapeutic activity are still needed.

### BRIEF DESCRIPTION OF THE FIGURES

FIG. 1. Design and general backbone of the new piperazine derivatives analogues of the hit compound 2.

FIG. 2. (A) Nuclear association of HAdV DNA. (B) Control for the specificity of nuclear DNA purification.

FIG. 3. Design and general backbone of the new piperazine derivatives analogues of the hit compound 2.

FIG. 4. Molecules derived from 482 and 499.

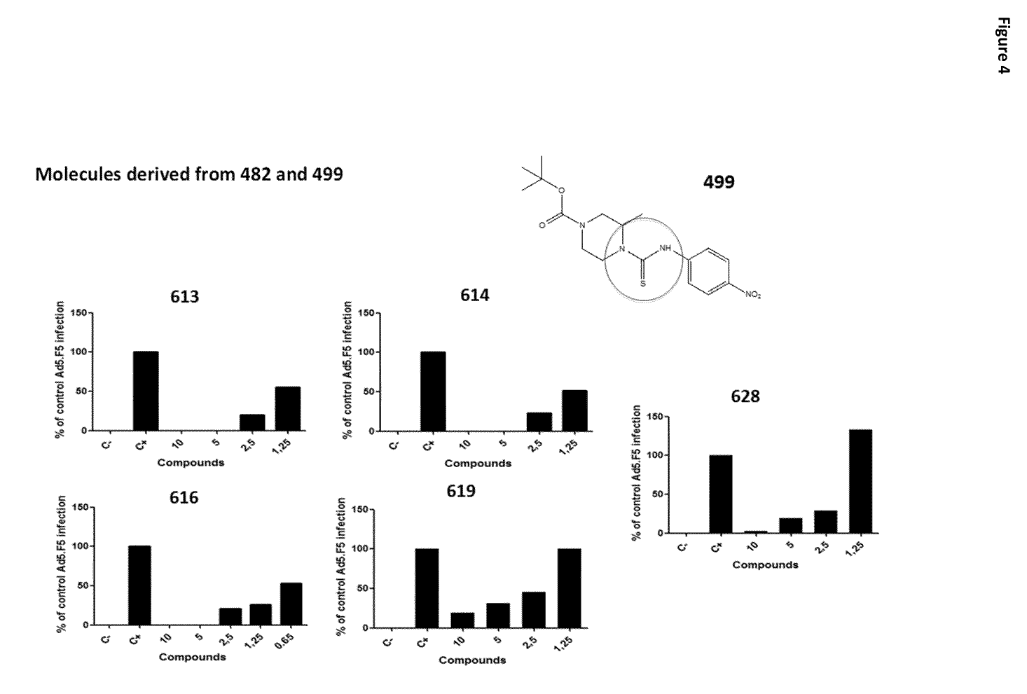

FIG. 5. Entry assays.

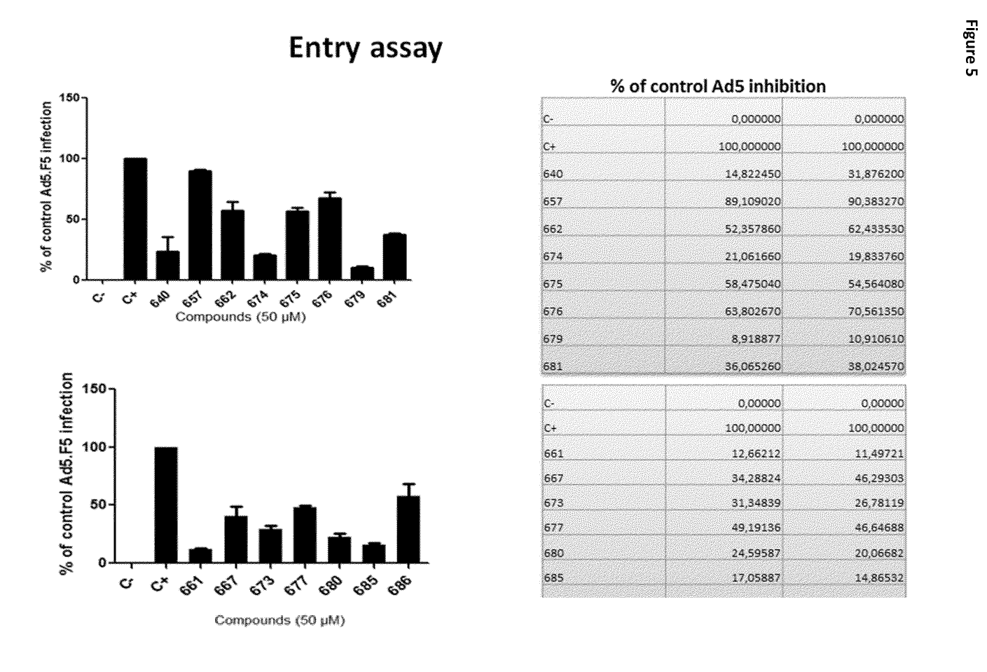

### BRIEF DESCRIPTION OF THE INVENTION

The present invention provides potential antiviral agents with increased therapeutic activity.

In particular these compounds have a chemical structure which comprises the following formula:

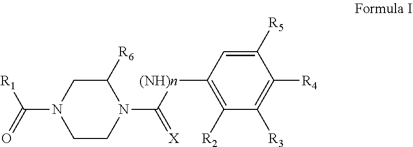

wherein

R1 is O^{t}Bu, ^{t}Bu, Ph, p-CH₃Ph, o-CH₃Ph, CH₂-^{t}Bu, CH₂-^{C}Hexyl, CH₂-Ph, CH═CHPh or Benzofuran-2-yl;

X is S or O;

R2 is H, NO₂, Cl, F, Br or OCH₃;

R3 is H or CF₃;

R4 is H, NO₂, Cl, F, CH₃, CN, CF₃ or OCH₃;

R5 is H or CF₃;

R6 is H, CH₃ or Ph; and

n is 0 or 1.

In addition, the present invention further provides potential antibacterial agents with increased therapeutic activity.

In particular these compounds have a chemical structure which comprises the following formula:

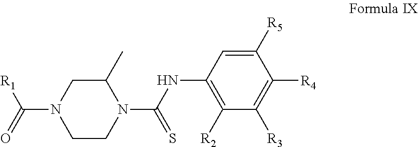

wherein

R1 is O^{t}Bu,CH₂^{t}Bu, CH₂Ph or CH₂^{C}Hexyl;

R2 is H;

R3 is H or CF₃;

R4 is NO₂, Cl, CN, F, CF₃ CH₃ or OCH₃; and

R5 is H or CF₃.

### DETAILED DESCRIPTION OF THE INVENTION

The major aim of this invention is to present the design, synthesis, by a short and high yielded methodology, and evaluation of three generations of new 4-acyl-1 phenylaminocarbonyl-2-methylpiperazine and 4-acyl-1 phenylaminocarbonyl-2 phenylpiperazine derivatives, 52 compounds in total. The authors have also established structure-activity relationships of these new compounds and identified 6 new 2-phenylpiperazine derivatives as potent inhibitors against HAdV and HCMV (human cytomegalovirus).

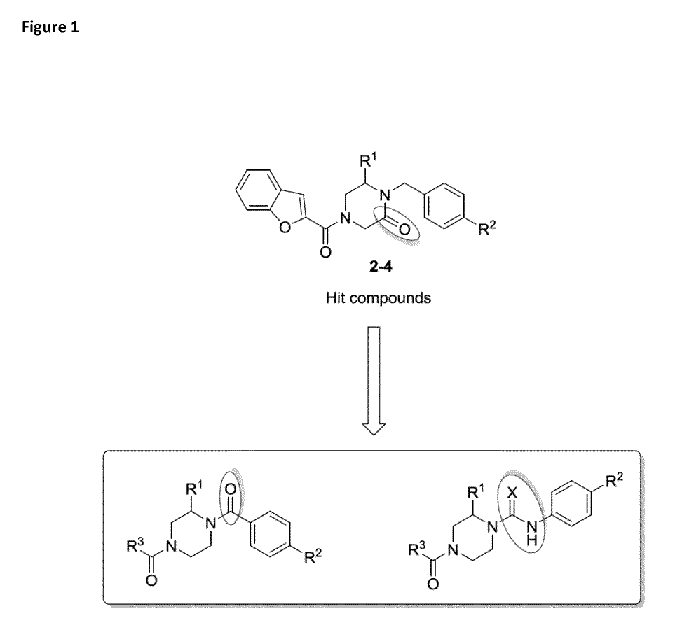

In order to achieve the results presented herein, we designed a general structure based on the structural modifications illustrated in FIG. 1 obtaining a general backbone with several structural variation points.

Based on the fact that piperazine derivatives had previously showed their utility as an effective source of antiviral compounds with different mechanisms of action, we have maintained this backbone as the fundamental core in our new compounds. The first structural modification made, that generates the new group of compounds, is the replacement of the piperazin-2-one ring by a piperazine one by moving the carbonyl group from the ring to the 1 nitrogen of the piperazine through an amide or urea/thiourea function. The presence of this exocyclic carbonyl group becomes the common feature of our new compounds.

In order to generate chemical diversity three points of variation in our general structure should be mentioned: (1) the substituent on the piperazine ring (R1); (2) the substituent of the new amide or urea functions at 1 nitrogen (R2 groups with different electronic properties); and (3) different acyl functions are located at the other nitrogen (urethane or amide groups with R3 an aryl or alkyl group).

Based on or derived from these modifications three different generations of compounds were created. The results for each family are indicated in examples 2 to 7. According to these results, it can be concluded that for 2-phenylpiperazine derivatives the presence of a benzofuran-2-yl group contributes to increase the antiviral activity as well as, for some substituents, the CC₅₀, giving more active and less cytotoxic compounds. In particular, it is worth mentioning that compounds 46, 59, 60, 63 and 64 cause a significant decrease in HAdV and HCMV DNA copy number and that activity could be the consequence of the inhibition of HAdV and HCMV DNA replication directly by interfering with a protein involved in this process or alternatively, these compounds may impact transcription of the immediate early genes, which is a pre-requisite for subsequent DNA replication.

Consequently, most of the compounds falling within the general formulae pertaining to each of the generations provided in examples 2 to 7, in particular compounds 46, 59, 60, 63, 64, and 65, have proven to be significant and broadspectrum inhibitors of DNA replication both in HAdV and HCMV. Therefore, although further optimization and characterization of their mechanisms of action will be required for these compounds, they represent strong hit candidates for the development of a new class of antiviral compounds.

Therefore, a first aspect of the invention refers to a composition comprising a compound having a chemical structure which comprises the following formula:

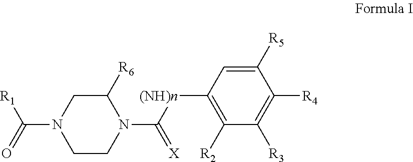

wherein

R1 is O^{t}Bu, ^{t}Bu, Ph, p-CH₃Ph, o-CH₃Ph, CH₂-^{t}Bu, CH₂-^{C}Hexyl, CH₂-Ph, CH═CHPh or Benzofuran-2-yl;

X is S or O;

R2 is H, NO₂, Cl, F, Br or OCH₃;

R3 is H or CF₃;

R4 is H, NO₂, Cl, F, CH₃, CN, CF₃ or OCH₃;

R5 is H or CF₃;

R6 is H, CH₃ or Ph; and

n is 0 or 1.

In a preferred embodiment of the first aspect of the invention, the compound comprises the following chemical structure:

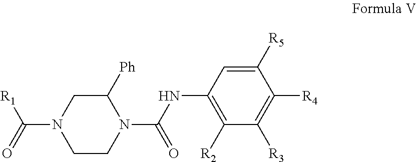

wherein

R1 is O^{t}Bu, ^{t}Bu, CH₂-^{t}Bu, Ph or Benzofuran-2-yl;

R2 is H, NO₂, Cl, F, Br or OCH₃;

R3 is H or CF₃;

R4 is H, NO₂, Cl, F, CH₃, CN, CF₃ or OCH₃; and

R5 is H or CF₃.

In another preferred embodiment of the first aspect of the invention, the compound comprises the following chemical structure:

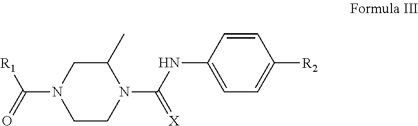

wherein

R1 is O^{t}Bu, ^{t}Bu, Ph or Benzofuran-2-yl;

R2 is NO₂ or OCH₃; and

X is S or O.

In another preferred embodiment of the first aspect of the invention, the compound comprises the following chemical structure:

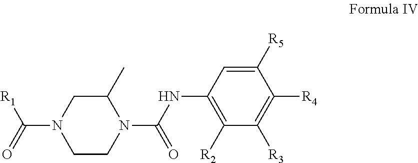

wherein

R1 is O^{t}Bu, ^{t}Bu, Ph or Benzofuran-2-yl;

R2 is H, NO₂, Cl, F, Br or OCH₃;

R3 is H or CF₃;

R4 is H, NO₂, Cl, F, CH₃, CN, CF₃ or OCH₃; and

R5 is H or CF₃.

In yet another embodiment of the first aspect of the invention, the compound comprises the following chemical structure:

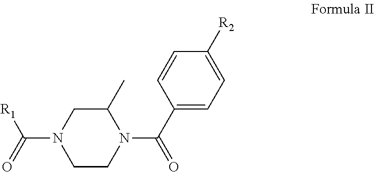

wherein

R1 is O^{t}Bu, ^{t}Bu, Ph or Benzofuran-2-yl; and

R2 is NO₂ or OCH₃.

In still another preferred embodiment of the first aspect of the invention, the compound comprises the following chemical structure:

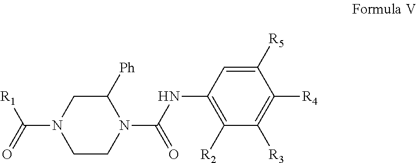

wherein

R1 is O^{t}Bu or Benzofuran-2-yl;

R2 is H, NO₂ or Cl;

R3 is H;

R4 is H, NO₂, Cl or CN; and

R5 is H or CF₃.

In still another preferred embodiment of the first aspect of the invention, the compound is selected from any of the following group of compunds consisting of:

a. A compound of formula V wherein R1 is O^{t}Bu; R2 is H; R3 is H; R4 is NO_{2;} and R5 is H;

b. A compound of formula V wherein R1 is Benzofuran-2-yl; R2 is H; R3 is H; R4 is NO_{2;} and R5 is H;

c. A compound of formula V wherein R1 is Benzofuran-2-yl; R2 is H; R3 is H; R4 is Cl; and R5 is H;

d. A compound of formula V wherein R1 is Benzofuran-2-yl; R2 is H; R3 is H; R4 is CN; and R5 is H;

e. A compound of formula V wherein R1 is Benzofuran-2-yl; R2 is NO_{2;} R3 is H; R4 is H; and R5 is H; and

f. A compound of formula V wherein R1 is Benzofuran-2-yl; R2 is Cl; R3 is H; R2 is H; and R5 is CF₃.

A second aspect of the invention refers to a pharmaceutical composition comprising a compound as defined in the first aspect of the invention or in any of its preferred embodiments, which further comprises pharmaceutically acceptable excipients, carriers or diluents.

A third aspect of the invention refers to a compound as defined in the first aspect of the invention or in any of its preferred embodiments, for use in therapy.

A fourth aspect of the invention refers to a compound as defined in the first aspect of the invention or in any of its preferred embodiments, for use in the treatment of an infection caused by a double-stranded DNA virus in a subject, preferably in a human subject. Prerably the double-stranded DNA virus is an adenovirus or herpesviruses. Preferably, the herpesvirus is a cytomegalovirus. Preferably, the double-stranded DNA virus is an adenovirus, more preferably selected from the group consisting of;

a. Specie A and types 12, 18, 31;

b. Specie B and types 3, 7, 11, 14, 16, 21, 34, 35, 50, 55;

c. Specie C and types 1, 2, 5, 6, 57;

d. Specie D and types 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54, 56;

e. Specie E and type 4;

f. Specie F and types 40 and 41; and

g. Specie G and type 52;

Still more preferably, the subject is a human subject suffering from a respiratory disease, form conjunctivitis, gastroenteritis, HIV, obesity or is subjected to immunosuppressive therapies.

In addition and as a second invention, the authors have departure from compound 2 shown below to present the design, synthesis, by a short and high yielded methodology, and evaluation of a new generation of compounds having anti-bacterial activity. The authors have also established structure-activity relationships of these new compounds and identified 4 new families of compounds having a significant antibacteridal activity.

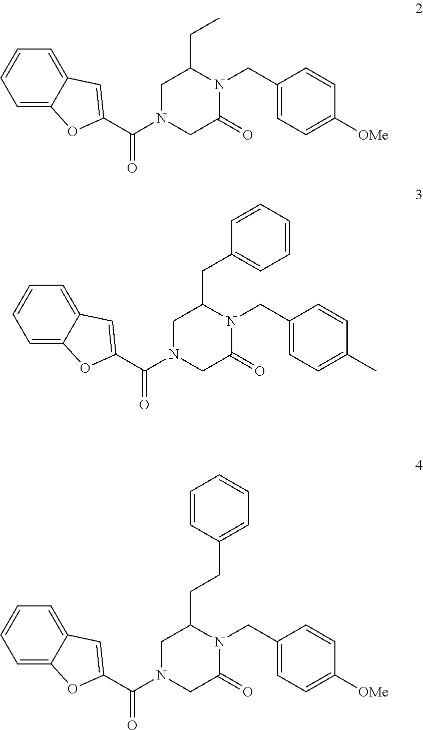 [SMILES: CCC1CN(C(=O)c2cc3ccccc3o2)CC(=O)N1Cc1ccc(OC)cc1 ; Cc1ccc(CN2C(=O)CN(C(=O)c3cc4ccccc4o3)CC2Cc2ccccc2)cc1 ; COc1ccc(CN2C(=O)CN(C(=O)c3cc4ccccc4o3)CC2CCc2ccccc2)cc1]

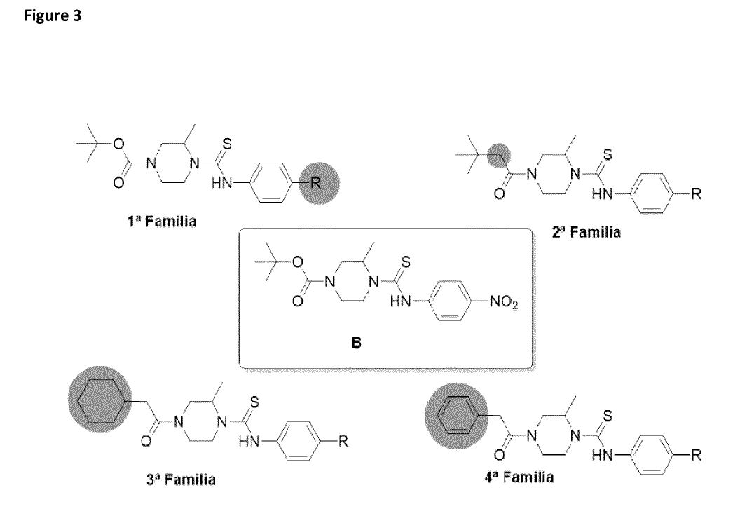

In this sense, the authors of the present invention have designed a general structure based on the structural modifications illustrated in FIG. 3 obtaining a general backbone with several structural variation points.

A first family of these types of compunds comprises the following general structure:

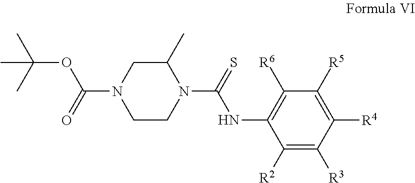

wherein

R₄ is NO₂, Cl, CN, F, CF₃, OCH₃, CH₃ or H;

R₃ is H or CF3;

R₅ is H or CF₃; and

wherein R₆ and R₂ are H.

A second family of these types of compounds comprises the following general structure:

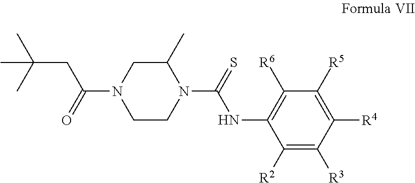

wherein

R₄ is NO₂, Cl, CN, F, CF₃, OCH₃, CH₃ or H;

R₃ is H or CF3;

R₅ is H or CF₃; and

wherein R₆ and R₂ are H.

A third family of these types of compunds comprises the following general structure:

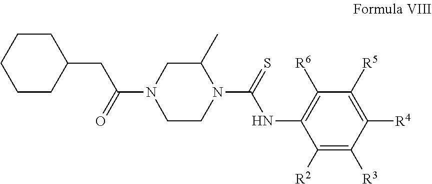

wherein

R₄ is NO₂, Cl, CN, F, CF₃, OCH₃, CH₃ or H;

R₃ is H or CF3;

R₅ is H or CF₃; and

wherein R₆ and R₂ are H.

A fourth family of these types of compunds comprises the following general structure:

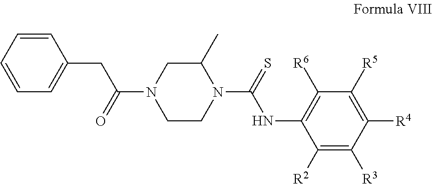

wherein

R₄ is NO₂, Cl, CN, F, CF₃, OCH₃, CH₃ or H;

R₃ is H or CF3;

R₅ is H or CF₃; and

wherein R₆ and R₂ are H.

These four different generations of compounds provided the results indicated in examples 9 to 16. According to these results, it can be concluded that these specific derivatives show an increased antibacterial activity, even for those cases of multiresistant bacteria such as A. baumannii colistine resistance bacteria.

Consequently, most of the compounds falling within the general formulae pertaining to each of the generations provided in examples 9 to 16 have proven to be significant and broadspectrum inhibitors of bacterial growth. Therefore, although further optimization and characterization of their mechanisms of action will be required for these compounds, they represent strong hit candidates for the development of a new class of antibacterial compounds.

Consequently, a first aspect of the second invention refers to a composition comprising a compound having a chemical structure which comprises the following formula:

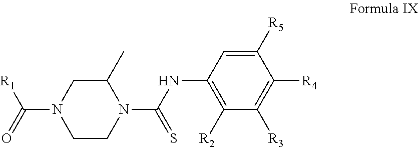

wherein

R1 is O^{t}Bu,CH₂^{t}Bu, CH₂Ph or CH₂^{C}Hexyl;

R2 is H;

R3 is H or CF₃;

R4 is NO₂, Cl, CN, F, CF₃ CH₃ or OCH₃; and

R5 is H or CF₃.

In a preferred embodiment of the first aspect of the second invention, the compound comprises the following general structure:

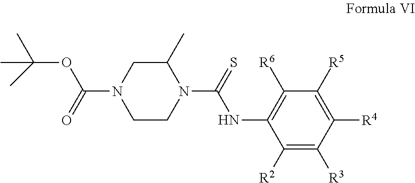

wherein

R₄ is NO₂, Cl, CN, F, CF₃, OCH₃, CH₃ or H;

R₃ is H or CF3;

R₅ is H or CF₃; and

wherein R₆ and R₂ are H.

In another preferred embodiment of the first aspect of the second invention, the compound comprises the following general structure:

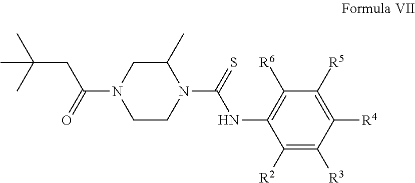

wherein

R₄ is NO₂, Cl, CN, F, CF₃, OCH₃, CH₃ or H;

R₃ is H or CF3;

R₅ is H or CF₃; and

wherein R₆ and R₂ are H.

In another preferred embodiment of the first aspect of the second invention, the compound comprises the following general structure:

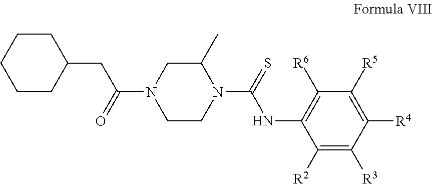

wherein

R₄ is NO₂, Cl, CN, F, CF₃, OCH₃, CH₃ or H;

R₃ is H or CF3;

R₅ is H or CF₃; and

wherein R₆ and R₂ are H.

In another preferred embodiment of the first aspect of the second invention, the compound comprises the following general structure:

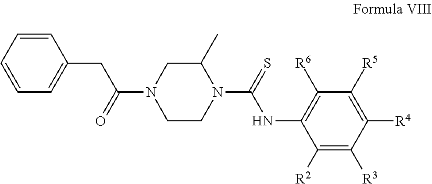

wherein

R₄ is NO₂, Cl, CN, F, CF₃, OCH₃, CH₃ or H;

R₃ is H or CF3;

R₅ is H or CF₃; and

wherein R₆ and R₂ are H.

In yet another preferred embodiment of the first aspect of the second invention, the compound is selected from the following list consisting of:

a. A compound of formula VI wherein R1 is O^{t}Bu; R2 is H; R3 is H; R4 is NO_{2;} and R5 is H.

b. A compound of formula VI wherein R1 is O^{t}Bu; R2 is H; R3 is H; R4 is Cl; and R5 is H.

c. A compound of formula VI wherein R1 is O^{t}Bu; R2 is H; R3 is H; R4 is CN; and R5 is H.

d. A compound of formula VI wherein R1 is O^{t}Bu; R2 is H; R3 is H; R4 is F; and R5 is H.

e. A compound of formula VI wherein R1 is O^{t}Bu; R2 is H; R3 is H; R4 is CF₃; and R5 is H.

f. A compound of formula VI wherein R1 is O^{t}Bu; R2 is H; R3 is H; R4 is OCH₃; and R5 is H.

g. A compound of formula VI wherein R1 is O^{t}Bu; R2 is H; R3 is H; R4 is CH₃; and R5 is H.

h. A compound of formula VI wherein R1 is O^{t}Bu; R2 is H; R3 is CF₃; R4 is H; and R5 is CF₃.

i. A compound of formula VI wherein R1 is CH₂^{t}Bu; R2 is H; R3 is H; R4 is NO₂; and R5 is H.

j. A compound of formula VI wherein R1 is CH₂^{t}Bu; R2 is H; R3 is H; R4 is Cl; and R5 is H.

k. A compound of formula VI wherein R1 is CH₂^{t}Bu; R2 is H; R3 is H; R4 is CN; and R5 is H.

l. A compound of formula VI wherein R1 is CH₂^{t}Bu; R2 is H; R3 is H; R4 is F; and R5 is H.

m. A compound of formula VI wherein R1 is CH₂^{t}Bu; R2 is H; R3 is H; R4 is CF₃; and R5 is H.

n. A compound of formula VI wherein R1 is CH₂^{t}Bu; R2 is H; R3 is H; R4 is OCH₃; and R5 is H.

n. A compound of formula VI wherein R1 is CH₂^{t}Bu; R2 is H; R3 is H; R4 is CH₃; and R5 is H.

o. A compound of formula VI wherein R1 is CH₂^{t}Bu; R2 is H; R3 is CF₃; R4 is H; and R5 is CF₃.

p. A compound of formula VI wherein R1 is CH₂^{C}Hexyl; R2 is H; R3 is H; R4 is NO₂; and R5 is H.

q. A compound of formula VI wherein R1 is CH₂^{C}Hexyl; R2 is H; R3 is H; R4 is Cl; and R5 is H.

r. A compound of formula VI wherein R1 is CH₂^{C}Hexyl; R2 is H; R3 is H; R4 is CN; and R5 is H.

s. A compound of formula VI wherein R1 is CH₂^{C}Hexyl; R2 is H; R3 is H; R4 is F; and R5 is H.

t. A compound of formula VI wherein R1 is CH₂^{C}Hexyl; R2 is H; R3 is H; R4 is CF₃; and R5 is H.

u. A compound of formula VI wherein R1 is CH₂^{C}Hexyl; R2 is H; R3 is H; R4 is OCH₃; and R5 is H.

v. A compound of formula VI wherein R1 is CH₂^{C}Hexyl h; R2 is H; R3 is H; R4 is CH₃; and R5 is H.

w. A compound of formula VI wherein R1 is CH₂^{C}Hexyl; R2 is H; R3 is CF₃; R4 is H; and R5 is CF₃.

x. A compound of formula VI wherein R1 is CH₂Ph; R2 is H; R3 is H; R4 is NO₂; and R5 is H.

y. A compound of formula VI wherein R1 is CH₂Ph; R2 is H; R3 is H; R4 is Cl; and R5 is H.

z. A compound of formula VI wherein R1 is CH₂Ph; R2 is H; R3 is H; R4 is CN; and R5 is H.

aa. A compound of formula VI wherein R1 is CH₂Ph; R2 is H; R3 is H; R4 is F; and R5 is H.

ab. A compound of formula VI wherein R1 is CH₂^{t}Bu; R2 is H; R3 is H; R4 is CF₃; and R5 is H.

ac. A compound of formula VI wherein R1 is CH₂Ph; R2 is H; R3 is H; R4 is OCH₃; and R5 is H.

ad. A compound of formula VI wherein R1 is CH₂Ph; R2 is H; R3 is H; R4 is CH₃; and R5 is H.

ae. A compound of formula VI wherein R1 is CH₂Ph; R2 is H; R3 is CF₃; R4 is H; and R5 is CF₃.

A second aspect of the second invention refers to a pharmaceutical composition comprising a compound as defined in the first aspect of the second invention or in any of its preferred embodiments, which further comprises pharmaceutically acceptable excipients, carriers or diluents.

A third aspect of the second invention refers to a compound as defined in the first aspect of the second invention or in any of its preferred embodiments, for use in therapy.

A fourth aspect of the invention refers to a compound as defined in the first aspect of the invention or in any of its preferred embodiments, for use in the treatment (prophylactic and/or therapeutic) of an infection caused in a subject, preferably a human subject, by a pathogenic bacteria such as a mycobacterium strain, preferably Mycobacterium tuberculosis, Escherichia coli, Pseudomonas aeruginosa or Klebsiella pneumoniae. Preferably such bacterium is a type of bacterium with a shape intermediate between cocci (spherical bacteria) and bacilli (rod-shaped bacteria). Examples of such bacterium are Haemophilus influenzae, Gardnerella vaginalis, and Chlamydia trachomatis. Other bacterium for which the present invention is useful is Aggregatibacter actinomycetemcomitans, Acinetobacter strains such as A. baumannii and Bordetella pertussis.

A fourth aspect of the invention refers to a compound as defined in the first aspect of the second invention or in any of its preferred embodiments, for use in the treatment of an infection caused by a bacteria resistant to colistin.

A fifth aspect of the invention refers to a compound as defined in the first aspect of the second invention or in any of its preferred embodiments, for use in the simultaneous or subsequent treatment of an infection caused by a bacteria with a polymyxin antibiotic such as colistin. Preferably such combination therapy is used for the treatment of colistin resistant bacterial strains. Preferably such strain is a mycobacterium strain, preferably Mycobacterium tuberculosis, Escherichia coli, Pseudomonas aeruginosa or Klebsiella pneumoniae. More preferably such bacterium is a type of bacterium with a shape intermediate between cocci (spherical bacteria) and bacilli (rod-shaped bacteria). Examples of such bacterium are Haemophilus influenzae, Gardnerella vaginalis, and Chlamydia trachomatis. Other bacterium for which the present invention is useful are Aggregatibacter actinomycetemcomitans, Acinetobacter strains such as A. baumannii and Bordetella pertussis. More preferably, such compound for use in the simultaneous or subsequent treatment of an infection caused by a bacteria with a polymyxin antibiotic such as colistin, is

[symbol]

Lastly, an additional aspect of the present invention refers to a process for the preparation of urea/thioureaderivatives of formula V and VI, which comprises the following steps:

a. to a solution of the 1-monoacyl derivatives adding isocyanate or isothiocyanate and stirring all starting materials react;

b. evaporate the solution of step a) to dryness, and

c. purified the compounds of step b) by flash chromatography on silica gel by using an appropriate eluent.

For the purpose of the present invention, the following definitions are included below:
The term “comprising” it is meant including, but not limited to, whatever follows the word “comprising”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of groups, those skilled in the art will recognize that the invention is also thereby described in terms of any individual member or subgroup of members of the group.

### EXAMPLES

### Example 1

### Materials and Methods

1.1. Chemistry. General Chemistry Methods. All reagents, solvents, and starting materials were obtained from commercial suppliers and used without further purification. The crude reaction mixtures were concentrated under reduced pressure by removing the organic solvents in a rotary evaporator. Reactions were monitored by thin layer chromatography (TLC) using Kieselgel 60 F254 (E. Merck) plates and UV detector for visualization. Flash column chromatography was performed on Silica Gel 60 (E. Merck) with the indicated eluent. All reported yields are of purified products. Melting points were obtained on a Stuart Melting Point Apparatus SMP 10 and are uncorrected. Mass spectra were recorded on a Micromass AUTOSPECQ mass spectrometer: EI at 70 eV and CI at 150 eV, HR mass measurements with resolutions of 10,000. FAB mass spectra were recorded using a thioglycerol matrix. NMR spectra were recorded at 25° C. on a Bruker AV500 spectrometer at 500 MHz for 1 H and 125 MHz for 13C). The chemical shifts (δ) reported are given in parts per million (ppm) and the coupling constants (J) are in hertz (Hz). 1H chemical shift values (δ) are referenced to the residual nondeuterated components of the NMR solvents (δ=2.54 ppm for DMSO, δ=7.26 ppm for CDCl3). The 13C chemical shifts (δ) are referenced to CDCl3 (central peak, ≡=77.16 ppm) as the internal standard. The spin multiplicities are reported as s (singlet), d (doublet), t (triplet), q (quadruplet), m (multiplet), or br s (broad singlet). COSY, DEPT, HSQC, and NOESY experiments were performed to assign the signals in the NMR spectra. The purity of final compounds was evaluated by C, H, N analysis. The purity of all the final compounds was confirmed to be ≥95% by combustion.

General Procedure 1. Chemoselective N-acylation reaction of 2-substitued piperazines (6-9, 43-45). 2-Substituted piperazine (6.0 mmol) was dissolved in dry dichloromethane (80 mL) and cooled to 0° C. A solution of the appropriate acylating agent in dichloromethane (6.0 mmol, 20 mL) was added dropwise in 30 minutes, and then pyridine (9 mmol). The reaction mixture was kept into an ice-water bath with stirring 12 hours and left at room temperature until TLC showed that all the starting material had reacted. The reaction mixture was evaporated to dryness to obtain the corresponding monoacylderivative. Column chromatography gave the pure compounds in high yields.

1-tert-Butoxycarbonyl-3-methylpiperazine (6).37. The product was obtained as a syrup and purified by column chromatography using dichloromethane-methanol (15:1) as eluent (0.90 g, 75% yield). MS (CI): m/z 201 (20%) [M+H]+. 1H NMR (500 MHz, DMSO-d6) δ 3.75-3.71 (m, 2H), 2.85-2.82 (m, 1H), 2.75-2.69 (m, 1H), 2.60-2.54 (m, 3H), 2.39-2.34 (m, 1H), 1.41 (s, 9H), 0.96 (d, J=6.3 Hz, 3H). 13C RMN (125 MHz, DMSO-d6) δ 154.5, 79.3, 51.2, 50.5, 45.5, 44.4, 28.6, 19.3. HRMS (m/z): calcd. for C10H20N2O2 200.1528 [M]+.; found 200.1525.

3-Methyl-1-pivaloylpiperazine (7).38. The product was obtained as a syrup and purified by column chromatography using dichloromethane-methanol (15:1) as eluent (0.85 g, 77% yield). MS (CI): m/z 185 (90%) [M+H]+. 1H NMR (500 MHz, DMSO-d6) δ 4.11-4.02 (m, 2H), 2.89-2.73 (m, 2H), 2.59-2.52 (m, 2H), 2.45-2.38 (m, 1H), 1.19 (s, 9H), 0.97 (d, J=6.3 Hz, 3H). 13C NMR (125 MHz, DMSO-d6) δ 176.9, 80.2, 51.2, 45.2, 44.6, 40.3, 28.6, 19.0.

1-Benzoyl-3-methylpiperazine (8).39. The product was obtained as a syrup and purified by column chromatography using dichloromethane-methanol (15:1) as eluent (1.0 g, 81% yield). MS (CI): m/z 205 (100%) [M+H]+. 1H NMR (500 MHz, DMSO-d6) δ 7.45-7.34 (m, 5H), 3.91-3.60 (m, 2H), 2.94-2.87 (m, 3H), 2.70-2.59 (m, 3H), 0.96 (d, J=5.8 Hz, 3H). 13C NMR (125 MHz, DMSO-d6) δ 168.8, 136.1, 128.9, 128.0, 126.4, 50.0, 44.9, 40.0, 18.5. HRMS (m/z): calcd. for C12H17N2O 205.1343 [M+H]+; found 205.1341.

1-(Benzofuran-2-carbonyl)-3-methylpiperazine (9). The product was obtained as a solid and purified by column chromatography using dichloromethane-methanol (40:1) as eluent (1.1 g, 74% yield), mp 101-103° C. 1H NMR (500 MHz, DMSO-d6) δ 7.7-7.5 (m, 5H), 4.47 (br s, 2H), 3.10 (d, J=11.4 Hz, 1H), 2.94-2.86 (m, 2H), 1.97 (br s, 2H), 1.13 (d, J=5.0 Hz, 3H). 13C NMR (125 MHz, DMSO-d6) δ 159.8, 154.6, 149.1, 127.0, 126.4, 123.6, 122.2, 111.9, 111.8, 51.1, 46.1, 19.4. Anal. calcd for C14H17N2O2: C, 68.55; H, 6.99; N, 11.42. Found: C, 68.32; H, 6.62; N, 11.22.

1-tert-Butoxycarbonyl-3-phenylpiperazine (43). The product was obtained as a syrup and purified by column chromatography using hexane-ethyl acetate (2:1) as eluent (1.04 g, 66% yield), mp 103-105° C. MS (CI): m/z 263 (100%) [M+H]+. 1H NMR (500 MHz, CDCl3) δ 7.4-7.3 (m, 5H), 4.05 (br s, 2H), 3.70 (dd, J=2.4 Hz, J=10.5 Hz, 1H,), 3.07 (m, 1H), 2.9-2.8 (m, 2H), 2.72 (br s, 1H), 1.90 (br s, 1H), 1.47 (s, 9H). 13C NMR (125 MHz, CDCl3) δ 154.8, 141.5, 128.5, 127.8, 127.0, 79,7 60.3, 51.5, 46.1, 43.4, 28.5. HRMS (m/z): calcd. for C15H23N2O2 263.1754 [M+H]+; found 263.1748.

3-Phenyl-1-pivaloylpiperazine (44). The product was obtained as a syrup and purified by column chromatography using dichloromethane-methanol (70:1) as eluent (1.36 g, 92% yield). MS (CI): m/z 247 (90%) [M+H]+. 1H NMR (500 MHz, CDCl3) δ 7.6-7.5 (m, 5H), 4.50 (d, J=13.8 Hz 1H), 4.38 (d, J=14.2 Hz, 1H), 3.93 (dd, J=2.9 Hz, J=11.2 Hz, 1H), 3.43 (t, J=13.4 Hz, 1H,), 3.36 (t, J=12.5 Hz, 1H), 3.05 (d, J=12.5 Hz, 1H), 2.84 (m, J=3.3 Hz, J=12.5 Hz), 1.27 (s, 9H). 13C NMR (125 MHz, CDCl3) δ 176.4, 134.5, 129.6, 129.3, 127.8, 60.1, 48.6, 44.5, 42.1, 38.8, 28.3.

1-(Benzofuran-2-carbonyl)-3-phenylpiperazine (45). The product was obtained as a syrup and purified by column chromatography using hexane-ethyl acetate (2:1) as eluent (1.21 g, 66% yield). MS (CI): m/z 307 (100%) [M+H]+. 1H NMR (500 MHz, DMSO-d6) δ 7.8-7.3 (m, 10H), 4.39 (br s, 2H), 3.78 (m, 1H,), 3.12 (m, 2H), 2.83 (dt, J=2.8 Hz, J=12.0 Hz, 2H). 13C NMR (125 MHz, CDCl3) δ 158.9, 153.9, 148.4, 141.7, 128.3, 127.5, 126.9, 126.7, 126.4, 123.7, 122.4, 111.7, 110.7, 59.8, 22.4. HRMS (m/z): calcd. for C19H19N2O2 307.1441 [M+H]+; found 307.1433. Anal. calcd for C19H18N2O2: C, 74.49; H, 5.92; N, 9.14. Found: C, 74.61; H, 6.18; N, 8.93.

General Procedure 2. Synthesis of the amide derivatives (10, 11, 15, 16, 20, and 21). To a solution of the 1-monoacyl derivatives (6-8) (1.0 mol) in dry dichloromethane (30 mL) was added the corresponding acyl halyde (1.5 mmol) and pyridine (1.5 mmol). The reaction mixture was stirred at room temperature until TLC showed that all the starting material had reacted. The reaction mixture was successively washed with diluted hydrochloric acid, aqueous saturated solution of sodium bicarbonate and water, dried (MgSO4), filtered and the filtrate was evaporated to dryness. The compounds obtained were purified by flash chromatography on silica gel.

4-tert-Butoxycarbonyl-2-methyl-1-(4-nitrobenzoyl)piperazine (10). The product was obtained as a solid and purified by column chromatography using hexane-ethyl acetate (2.5:1) as eluent (181 mg, 52% yield), mp 115-117° C. MS (CI): m/z 350 (30%) [M+H]+. 1H NMR (500 MHz, DMSO-d6) δ 8.30-8.27 (m, 2H), 7.68-7.65 (m, 2H), 3.95-3.88 (m, 1H), 3.79-3.73 (m, 2H), 3.13-3.09 (m, 2H), 2.95-2.88 (m, 2H), 1.44 (s, 9H), 1.18 (d, J=6.8 Hz, 3H). 13C RMN (125 MHz, DMSO-d6) δ 168.3, 151,1, 141.9, 128.5, 124.3, 79.8, 51.1, 44.2, 39.4, 28.6, 15.7. HRMS (m/z): calcd. for C17H23N3O5 349.1637 [M]+.; found 349.1638. Anal. calcd for C17H23N3O5: C, 58.44; H, 6.64; N, 12.03. Found: C, 58.62; H, 6.75; N, 11.84.

4-tert-Butoxycarbonyl-1-(4-methoxybenzoyl)-2-methylpiperazine (11). The product was obtained as a solid and purified by column chromatography using hexane-ethyl acetate (4:1) as eluent (237 mg, 71% yield), mp 84-87° C. MS (CI): m/z 335 (28%) [M+H]+. 1H NMR (500 MHz, DMSO-d6) δ 7.38-7.33 (m, 2H), 7.01-6.98 (m, 2H), 4.35-4.28 (m, 1H), 3.93-3.83 (m, 3H), 3.82 (s, 3H), 3.78-3.73 (m, 1H), 2.91-2.84 (m, 2H), 1.43 (s, 9H), 1.15 (d, J=6.7 Hz, 3H). 13C RMN (125 MHz, DMSO-d6) δ 160.8, 155.1, 129.2, 114.5, 79.8, 55.9, 48.0, 43.9, 32.4, 28.5, 15.6. HRMS (m/z): calcd. for C18H26N2O4 334.1890 [M]+.; found 334.1893. Anal. calcd for C18H26N2O4: C, 64.65; H, 7.84; N, 8.38. Found: C, 64.77; H, 7.42; N, 8.33.

2-Methyl-1-(4-nitrobenzoyl)-4-pivaloylpiperazine (15). The product was obtained as a solid and purified by column chromatography using hexane-ethyl acetate (1.5:1) as eluent (173 mg, 52% yield), mp 143-145° C. MS (CI): m/z 334 (70%) [M+H]+. 1H NMR (500 MHz, DMSO-d6) δ 8.30-8.27 (m, 2H), 7.69-7.66 (m, 2H), 4.38-4.13 (m, 3H), 3.93-3.67 (m, 1H), 3.12-3.03 (m, 3H), 1.24 (s, 9H), 1.17 (d, J=6.7 Hz, 3H). 13C RMN (125 MHz, DMSO-d6) δ 177.1, 168.1, 144.8, 143.0, 128.7, 124.3, 80.2, 51.1, 48.8, 45.4, 38.8, 28.7, 15.8. FIRMS (m/z): calcd. for C17H24N3O4 334.1770 [M+H]+; found 334.1767. Anal. calcd for C17H23N3O4: C, 61.25; H, 6.95; N, 12.60. Found: C, 61.08; H, 7.01; N, 12.43.

1-(4-Methoxybenzoyl)-2-methyl-4-pivaloylpiperazine (16). The product was obtained as a solid and purified by column chromatography using hexane-ethyl acetate (2:1) as eluent (194 mg, 61% yield), mp 96-97° C. MS (CI): m/z 319 (35%) [M+H]+. 1H NMR (500 MHz, DMSO-d6) δ 7.38-7.35 (m, 2H), 7.01-6.99 (m, 2H), 4.41-4.31 (m, 1H), 4.23-4.12 (m, 2H), 3.92-3.85 (m, 1H), 3.82 (s, 3H), 3.09-2.99 (m, 3H), 1.24 (s, 9H), 1.14 (d, J=6,9 Hz, 3H). 13C RMN (125 MHz, DMSO-d6) δ 129.4, 114.3, 80.2, 55.8, 48.4, 45.7, 38.8, 28.7, 27.4, 15.8. HRMS (m/z): calcd. for C18H27N2O3 319.2016 [M+H]+; found 319.2022. Anal. calcd for C18H26N2O3: C, 67.90; H, 8.23; N, 8.80. Found: C, 67.55; H, 8.17; N, 8.49.

4-Benzoyl-2-methyl-1-(4-nitrobenzoyl)piperazine (20). The product was obtained as a solid and purified by column chromatography using hexane-ethyl acetate (1:2) as eluent (205 mg, 58% yield), mp 103-105° C. MS (CI): m/z 354 (85%) [M+H]+. 1H NMR (500 MHz, DMSO-d6) 8.30-8.27 (m, 2H), 7.70-7.67 (m, 2H), 7.48-7.40 (m, 5H), 4.40-4.22 (m, 1H), 4.10-3.92 (m, 1H), 3.89-3.70 (m, 2H), 1.20 (d, J=6.7 Hz, 3H). 13C NMR (125 MHz, CDCl3) δ 171.2, 156.8, 129.2, 114.5, 47,7, 15.9. FIRMS (m/z): calcd. for C19H20N3O4 354.1457 [M+H]+; found 354.1454.

4-Benzoyl-1-(4-methoxybenzoyl)-2-methylpiperazine (21). The product was obtained as a solid and purified by column chromatography using hexane-ethyl acetate (3:1) as eluent (210 mg, 62% yield). MS (CI): m/z 339 (20%) [M+H]+. 1H NMR (500 MHz, DMSO-d6) δ 8.31-8.25 (m, 2H), 7.72-7.68 (m, 2H), 7.44-7.39 (m, 5H), 4.35-4.28 (m, 1H), 3.93-3.83 (m, 3H), 3.82 (s, 3H), 3.78-3.73 (m, 1H), 2.91-2.84 (m, 2H), 1.15 (d, J=6.7 Hz, 3H). 13C NMR (125 MHz, CDCl3) δ 171.2, 155.1, 136.4, 133.9, 129.9, 128.9, 127.3, 122.6, 114.5, 55.9, 48.0, 43.9, 32.4, 28.5, 15.6. HRMS (m/z): calcd. for C20H23N2O3 339.1709 [M+H]+; found 339.1701.

General Procedure 3. Synthesis of the urea/thiourea derivatives (12-14, 17-19, 22-41, 46-65). To a solution of the 1-monoacyl derivatives (6-9, 43-45) (1.0 mol) in dry dichloromethane (20 mL) was added the corresponding isocyanate or isothiocyanate (1.2 mmol). The reaction mixture was stirred at room temperature until TLC showed that all the starting material had reacted. The reaction mixture was evaporated to dryness. The compounds were purified by flash chromatography on silica gel using the appropriate eluent.

4-tert-Butoxycarbonyl-2-methyl-1-[(4-nitrophenyl)aminothiocarbonyl]piperazine (12). The product was obtained as a solid and purified by column chromatography using hexane-ethyl acetate (2:1) as eluent (289 mg, 76% yield), mp 174-176° C. MS (FAB): m/z 403 (95%) [M+Na]+. 1H NMR (500 MHz, DMSO-d6) δ 9.42 (br s, 1H), 8.16-8.11 (m, 2H), 7.61-7.59 (m, 2H), 5.14-5.05 (m, 1H), 4.43-4.35 (m, 1H), 3.92-3.76 (m, 2H), 3.42-3.34 (m, 1H), 3.04-2.9 7 (m, 2H), 1.44 (s, 9H), 1.21 (d, J=6.7 Hz, 3H). 13C RMN (125 MHz, DMSO-d6) δ 181.3, 154.5, 147.7.0, 142.4, 123.9, 123.2, 79.6, 51.8, 43.0, 27.9, 14.7. HRMS (m/z): calcd. for C17H24N4O4SNa 403.1410 [M+Na]+; found 403.1405. Anal. calcd C17H24N4O4S: C, 53.67; H, 6.36; N, 14.73; S, 8.43. Found: C, 53.60; H, 6.58; N, 14.56; S, 8.27.

4-tert-Butoxycarbonyl-2-methyl-1-[(4-nitrophenyl)aminocarbonyl]piperazine (13). The product was obtained as a solid and purified by column chromatography using hexane-ethyl acetate (2:1) as eluent (266 mg, 73% yield), mp 165-167° C. MS (FAB): m/z 387 (45%) [M+Na]+. 1H NMR (500 MHz, DMSO-d6) δ 8.15-8.12 (m, 2H), 7.96-7.93 (m, 1H), 7.74-7.70 (m, 2H), 4.43-4.35 (m, 1H), 3.94-3.86 (m, 2H), 3.78-3.71 (m, 3H), 3.13-3.08 (m, 1H), 1.45 (s, 9H), 1.15 (d, J=6.6 Hz, 3H). 13C RMN (125 MHz, DMSO-d6) δ 155.2, 154.3, 147.8, 142.1, 125.0, 119.3, 79.7, 55.1, 47.2, 43.7, 39.0, 28.6, 15.6. HRMS (m/z): calcd. for C17H24N4O5Na 387.1639 [M+Na]+; found 387.1631. Anal. calcd C17H24N4O5: C, 56.03; H, 6.64; N, 15.38. Found: C, 55.99; H, 6.80; N, 15.28.

4-tert-Butoxycarbonyl-1-[(4-methoxyphenyl)aminocarbonyl]-2-methylpiperazine (14). The product was obtained as a solid and purified by column chromatography using hexane-ethyl acetate (2:1) as eluent (168 mg, 48% yield), mp 184-186° C. MS (CI): m/z 350 (10%) [M+H]+. 1H NMR (500 MHz, DMSO-d6) δ 8.01 (br s, 1H), 7.34-7.31 (m, 2H), 6.85-6.81 (m, 2H), 4.35-4.29 (m, 1H), 3.91-3.86 (m, 1H), 3.85-3.80 (m, 1H), 3.76-3.74 (m, 2H), 3.76-3.72 (m, 2H), 1.45 (s, 9H), 1.11 (d, J=6.6 Hz, 3H). 13C RMN (125 MHz, DMSO-d6) δ 156.0, 155.5, 134.0, 122.7, 116.8, 114.3, 79.5, 55.8, 46.7, 43.8, 38.7, 28.6, 15.4. HRMS (m/z): calcd. for C18H27N3O4 349.2005 [M]+.; found 349.2002. Anal. calcd C18H27N3O4: C, 61.87; H, 7.79; N, 12.03. Found: C, 61.60; H, 7.82; N, 11.99.

2-Methyl-1-[(4-nitrophenyl)aminothiocarbonyl]-4-pivaloylpiperazine (17). The product was obtained as a solid and purified by column chromatography using hexane-ethyl acetate (1:1) as eluent (222 mg, 61% yield), mp 168-170° C. MS (FAB): m/z 387 (100%) [M+Na]+. 1H NMR (500 MHz, DMSO-d6) δ 9.77 (br s, 1H), 8.17-8.11 (m, 2H), 7.63-7.58 (m, 2H), 5.15-5.05 (m, 1H), 4.47-4.38 (m, 1H), 4.23-4.11 (m, 2H), 3.23-3.10 (m, 3H), 1.25 (s, 9H), 1.1 (d, J=6.3 Hz, 3H). 13C RMN (125 MHz, DMSO-d6) δ 182.3, 124.3, 122.5, 53.5, 48.7, 45.1, 43.5 40.5, 28.6, 15.6. HRMS (m/z): calcd. for C17H24N4O3SNa 387.1461 [M+Na]+; found 387.1455. Anal. calcd for C17H24N4O3S: C, 56.02; H, 6.64; N, 15.37; S, 8.80. Found: C, 55.83; H, 6.68; N, 15.07; S, 8.64.

2-Methyl-1-[(4-nitrophenyl)aminocarbonyl]-4-pivaloylpiperazine (18). The product was obtained as a solid and purified by column chromatography using hexane-ethyl acetate (2:1) as eluent (237 mg, 68% yield), mp 220-222° C. MS (CI): m/z 349 (18%) [M+H]+. 1H NMR (500 MHz, DMSO-d6) δ 8.80 (br s, 1H), 8.15-8.12 (m, 2H), 7.74-7.71 (m, 2H), 4.47-4.38 (m, 1H), 4.20-4.12 (m, 2H), 3.97-3.92 (m, 1H), 3.14-3.06 (m, 3H), 1.26 (s, 9H), 1.14 (d, J=6.7 Hz, 3H). 13C RMN (125 MHz, DMSO-d6) δ 176.9, 154.5, 147.8, 141.9, 125.2, 119.1, 80.2, 51.1, 48.3, 47.7, 45.4 39.4, 28.6, 15.7. FIRMS (m/z): calcd. for C17H25N4O4 349.1866 [M+H]+; found 349.1876. Anal. calcd C17H24N4O4: C, 58.61; H, 6.94; N, 16.08. Found: C, 58.44; H, 6.98; N, 16.02.

1-[(4-Methoxyphenyl)aminocarbonyl]-2-methyl-4-pivaloylpiperazine (19). The product was obtained as a solid and purified by column chromatography using hexane-ethyl acetate (1:2) as eluent (197 mg, 59% yield), mp 171-173° C. MS (CI): m/z 334 (12%) [M+H]+. 1H NMR (500 MHz, DMSO-d6) δ 8.11 (br s, 1H), 8.15-8.12 (m, 2H), 7.35-7.32 (m, 2H), 6.85-7.82 (m, 2H), 4.40-4.32 (m, 1H), 4.18-4.05 (m, 2H), 3.91-3.86 (m, 1H), 3.73 (s, 3H), 3.13-3.03 (m, 3H), 1.26 (s, 9H), 1.10 (d, J=6.5 Hz, 3H). 13C RMN (125 MHz, DMSO-d6) δ 177.7, 155.7, 134.0, 122.7, 121.0, 114.3, 79.8, 55.9, 48.5, 47.4, 45.6 39.1, 28.7, 15.6. FIRMS (m/z): calcd. for C18H27N3O3 333.2056 [M]+.; found 333.2052. Anal. calcd C18H27N3O3: C, 64.84; H, 8.16; N, 12.60. Found: C, 64.67; H, 8.05; N, 12.33.

4-Benzoyl-2-methyl-1-[(4-nitrophenyl)aminothiocarbonyl]piperazine (22). The product was obtained as a solid and purified by column chromatography using hexane-ethyl acetate (2:1) as eluent (250 mg, 65% yield). MS (CI): m/z 385 (30%) [M+H]+. 1H NMR (500 MHz, DMSO-d6) δ 8.22-8.14 (m, 2H), 7.95-7.92 (m, 1H), 7.64-7.41 (m, 2H), 7.50-7.44 (m, 5H), 6.66-6.64 (m, 1H), 6.41 (br s, 1H), 5.16 (bs, 1H), 4.47-4.45 (m, 1H), 4.08-4.02 (m, 2H), 3.50-3.38 (m, 2H), 2.86-2.65 (m, 1H) 1.25 (d, J=6.6 Hz, 3H). 13C RMN (125 MHz, DMSO-d6) δ 182.7, 155.3, 148.3, 143.2, 136.1, 128.9, 127.4, 126.6, 124.2, 123.6, 55.1, 43.6, 40.7, 40.5, 15.5. Anal. calcd for C19H20N4O3S: C, 59.36; H, 5.24; N, 14.57; S, 8.34. Found: C, 59.19; H, 4.96; N, 14.35; S, 8.57.

4-Benzoyl-2-methyl-1-[(4-nitrophenyl)aminocarbonyl]piperazine (23). The product was obtained as a solid and purified by column chromatography using hexane-ethyl acetate (1:3) as eluent (339 mg, 92% yield), mp 208-210° C. MS (CI): m/z 369 (20%) [M+H]+. 1H NMR (500 MHz, DMSO-d6) δ 8.20 (br s, 1H), 8.15-8.12 (m, 2H), 7.74-7.70 (m, 2H), 7.49-7.42 (m, 5H), 4.47-4.44 (m, 1H), 4.06-3.80 (m, 2H), 1.17 (d, J=6.4 Hz, 3H). 13C RMN (125 MHz, DMSO-d6) δ 166.0, 154.5, 147.6, 130.4, 128.9, 127.4, 126.6, 124.4, 119.3, 113.1, 47.7, 39.2, 15.6. HRMS (m/z): calcd. for C19H21N4O4 369.1556 [M+H]+; found 369.1563. Anal. calcd for C19H20N4O4: C, 61.95; H, 5.47; N, 15.21. Found: C, 61.78; H, 5.57; N, 15.33.

4-Benzoyl-1-[(4-methoxyphenyl)aminocarbonyl]-2-methylpiperazine (24). The product was obtained as a solid and purified by column chromatography using hexane-ethyl acetate (1:2) as eluent (251 mg, 71% yield), mp 171-173° C. MS (CI): m/z 354 (15%) [M+H]+. 1H NMR (500 MHz, DMSO-d6) δ 8.20 (br s, 1H), 7.49-7.31 (m, 6H), 6.84-6.82 (m, 2H), 4.38 (m, 1H), 3.91-3.87 (m, 3H), 3.73 (s, 3H), 3.31-3.23 (m, 2H), 1.13 (d, J=6.4 Hz, 3H). 13C RMN (125 MHz, DMSO-d6) δ 170.8, 155.8, 136.4, 133.9, 129.9, 128.9, 127.3, 122.6, 114.3, 79.5, 55.9, 47.1 38.8, 28.6, 15.5. HRMS (m/z): calcd. for C20H23N3O3 353.1737 [M]+.; found 353.1739. Anal. calcd for C20H23N3O3: C, 67.97; H, 6.56; N, 11.89. Found: C, 67.72; H, 6.63; N, 11.82.

4-(Benzofuran-2-carbonyl)-2-methyl-1-[(4-nitrophenyl)aminocarbonyl]piperazine (25). The product was obtained as a solid and purified by column chromatography using hexane-ethyl acetate (1.5:1) as eluent (241 mg, 59% yield), mp 99-101° C. MS (FAB): m/z 431 (40%) [M+Na]+. 1H NMR (500 MHz, DMSO-d6) δ 9.23 (s, 1H), 8.2-7.7 (m, 9H), 4.47 (s, 1H), 4.34 (d, J=11.6 Hz, 1H), 4.4-4.3 (m, 2H), 3.3-3.2 (m, 2H), 1.16 (d, J=6.7 Hz, 3H). 13C RMN (125 MHz, DMSO-d6) δ 159.8, 154.1, 153.9, 147.0, 141.1, 126.8, 126.5, 125.1, 124.6, 123.8, 122.6, 118.8, 111.8, 111.4, 59.9, 46.9, 15.4. FIRMS (m/z): calcd. for C21H20N4O5Na 431.1326

[M+Na]+; found 431.1316. Anal. calcd C21H20N4O5: C, 61.76; H, 4.94; N, 13.72. Found: C, 62.03; H, 5.06; N, 13.39.

4-(Benzofuran-2-carbonyl)-1-[(4-methoxyphenyl)aminocarbonyl]-2-methylpiperazine (26). The product was obtained as a solid and purified by column chromatography using hexane-ethyl acetate (3:1) as eluent (330 mg, 84% yield), mp 170-172° C. MS (FAB): m/z 416 (100%) [M+Na]+. 1H NMR (500 MHz, DMSO-d6) δ 8.39 (s, 1H), 7.8-6.8 (m, 9H), 4.4-4.3 (m, 3H), 4.18 (m, 2H), 3.70 (s, 3H), 3.20 (t, J=2.8 Hz, J=11.2 Hz, 2H), 1.13 (d, J=6.5 Hz, 3H). 13C RMN (125 MHz, DMSO-d6) δ 155.9, 155.3, 154.5, 148.3, 133.3, 127.3, 127.1, 124.4, 123.1, 122.9, 120.9, 114.1, 112.3, 111.9, 55.7, 55.6, 47.2, 15.4. HRMS (m/z): calcd. for C22H23N3O4Na 416.1581 [M+Na]+; found 416.1576. Anal. calcd C22H23N3O4: C, 67.16; H, 5.89 N, 10.68. Found: C, 67.18; H, 5.75; N, 10.62.

4-tert-Butoxycarbonyl-1-[(4-chlorophenyl)aminocarbonyl]-2-methylpiperazine (27). The product was obtained as a solid and purified by column chromatography using hexane-ethyl acetate (5:1) as eluent (247 mg, 70% yield), mp 174-176° C. MS (FAB): m/z 376 (100%) [M+Na]+. 1H NMR (500 MHz, DMSO-d6) δ 8.60 (s, 1H), 7.41 (d, J=9.1 Hz, 2H), 7.26 (d, J=9.1 Hz, 2H), 4.29 (br s, 1H), 3.02 (dt, J=3.6 Hz, J=12.9 Hz, 2H), 2.84 (br s, 1H), 1.40 (s, 9H), 1.09 (d, J=6.7 Hz, 3H). 13C RMN (125 MHz, DMSO-d6) δ 154.8, 154.5, 139.0, 128.2, 125.8, 121.6, 79.4, 46,4, 27.9, 14.7. HRMS (m/z): calcd. for C17H24C1N3O3Na 376.1398 [M+Na]+; found 376.1389. Anal. calcd C17H24C1N3O3: C, 57.70; H, 6.84; N, 11.88. Found: C, 57.79; H, 6.58; N, 11.70.

4-tert-Butoxycarbonyl-2-methyl-1-[(4-trifluoromethylphenyl)aminocarbonyl]piperazine (28). The product was obtained as a solid and purified by column chromatography using hexane-ethyl acetate (5:1) as eluent (221 mg, 57% yield), mp 187-189° C. MS (FAB): m/z 410 (100%) [M+Na]+. 1H NMR (500 MHz, DMSO-d6) δ 8.86 (s, 1H), 7.60 (d, J=8.8 Hz, 2H), 7.56 (d, J=8.8 Hz, 2H), 4.30 (br s, 1H), 3.05 (dt, J=3.6 Hz, J=12.8 Hz, 2H), 2.84 (br s, 1H), 1.40 (s, 9H), 1.08 (d, J=6.8 Hz, 3H). 13C RMN (125 MHz, DMSO-d6) δ 155.2, 155.1, 144.5, 126.1, 123.9, 119.9, 80.0, 47.1, 28.5, 15.3. HRMS (m/z): calcd. for C18H24F3N3O3Na 410.1662 [M+Na]+; found 410.1652. Anal. calcd C18H24F3N3O3: C, 55.81; H, 6.24; N, 10.85. Found: C, 56.03; H, 6.27; N, 10.76.

4-tert-Butoxycarbonyl-2-methyl-1-[(2-nitrophenyl)aminocarbonyl]piperazine (29). The product was obtained as a solid and purified by column chromatography using hexane-ethyl acetate (5:1) as eluent (345 mg, 95% yield), mp 199-201° C. MS (FAB): m/z 387 (100%) [M+Na]+. 1H NMR (500 MHz, DMSO-d6) δ 9.29 (br s, 1H), 7.97-7.95 (m, 1H), 7.71-7.64 (m, 2H), 7.25-7.21 (m, 1H), 4.29 (br s, 1H), 3.94 (br s, 1H), 3.85-3.77 (m, 2H), 3.13-3.07 (m, 2H), 2.84 (br s, 1H), 1.44 (s, 9H), 1.15 (d, J=6.6 Hz, 3H). 13C RMN (125 MHz, DMSO-d6) δ 155.2, 155.2, 134.6, 134.0, 125.0, 123.8, 122.9, 119.1, 79.0, 46.9, 38.3, 28.0, 15.0. HRMS (m/z): calcd. for C17H24N4O5Na 387.1639 [M+Na]+; found 387.1628. Anal. calcd C17H24N4O5: C, 56.03; H, 6.64; N, 15.38. Found: C, 56.13; H, 6.42; N, 15.47.

4-tert-Butoxycarbonyl-1-[(2-chloro-5-trifluoromethylphenyl)aminocarbonyl]-2-methylpiperazine (30). The product was obtained as a solid and purified by column chromatography using hexane-ethyl acetate (7.5:1) as eluent (396 mg, 94% yield), mp 98-100° C. MS (FAB): m/z 444 (100%) [M+Na]+. 1H NMR (500 MHz, DMSO-d6) δ 8.41 (br s, 1H), 7.94-7.93 (m, 1H), 7.73-7.71 (m, 2H), 7.51-7.49 (m, 1H), 4.33 (br s, 1H), 3.92 (br s, 1H), 3.86-3.76 (m, 2H), 3.12-3.07 (m, 2H), 2.90 (br s, 1H), 1.44 (s, 9H), 1.15 (d, J=6.6 Hz, 3H). 13C RMN (125 MHz, DMSO-d6) δ 154.4, 154.3, 137.6, 131.6, 130.4, 127.7, 122.4, 121.6, 79.0, 46.8, 38.3, 28.0, 14.8. HRMS (m/z): calcd. for C18H23C1F3N3O3Na 444.1272 [M+Na]+; found 444.1259. Anal. calcd C18H23C1F3N3O3: C, 51.25; H, 5.50; N, 9.96. Found: C, 51.47; H, 5.64; N, 9.72

4-tert-Butoxycarbonyl-1-[(4-chloro-3-trifluoromethylphenyl)aminocarbonyl]-2-methylpiperazine (31). The product was obtained as a solid and purified by column chromatography using hexane-ethyl acetate (5:1) as eluent (404 mg, 96% yield), mp157-159° C. MS (FAB): m/z 444 (100%) [M+Na]+. 1H NMR (500 MHz, DMSO-d6) δ 8.91 (br s, 1H), 8.05 (m, 1H), 7.81-7.79 (m, 2H), 7.59-7.57 (m, 1H), 4.36 (br s, 1H), 4.00-3.75 (m, 3H), 3.10-3.04 (m, 2H), 2.87 (br s, 1H), 1.44 (s, 9H), 1.11 (d, J=7.1 Hz, 3H). 13C RMN (125 MHz, DMSO-d6) δ 154.2, 140.2, 131.6, 130.4, 126.2, 124.0, 122.2, 118.0, 79.0, 46.4, 38.2, 28.2, 14.9. HRMS (m/z): calcd. for C18H23C1F3N3O3Na 444.1272 [M+Na]+; found 444.1267. Anal. calcd C18H23C1F3N3O3: C, 51.25; H, 5.50; N, 9.96. Found: C, 51.33; H, 5.29; N, 9.63.

4-(Benzofuran-2-carbonyl)-1-[(4-chlorophenyl)aminocarbonyl]-2-methylpiperazine (32). The product was obtained as a solid and purified by column chromatography using hexane-ethyl acetate (1:1) as eluent (334 mg, 84% yield), mp 97-99° C. MS (FAB): m/z 420 (100%) [M+Na]+. 1H NMR (500 MHz, DMSO-d6) δ 8.65 (s, 1H), 7.7-7.3 (m, 9H), 4.43 (s, 1H), 4.32 (d, J=11.3 Hz 1H), 4.19 (m, 2H), 3.3-3.2 (m, 2H), 1.14 (d, J=6.6 Hz). 13C RMN (125 MHz, DMSO-d6) δ 159.8, 154.7, 153.9, 147.7, 139.0, 128.2, 126.8, 126.5, 125.9, 123.8, 122.6, 121.6, 111.8, 111.4, 59.5, 48.7, 46.7, 14.9. HRMS (m/z): calcd. for C21H20C1N3O3Na 420.1085 [M+Na]+; found 420.1079. Anal. calcd C21H20C1N3O3: C, 63.40; H, 5.07; N, 10.56. Found: C, 63.79; H, 5.13; N, 10.22.

4-(Benzofuran-2-carbonyl)-2-methyl-1-[(4-trifluoromethylphenyl)aminocarbonyl]piperazine (33). The product was obtained as a solid and purified by column chromatography using hexane-ethyl acetate (1.5:1) as eluent (267 mg, 62% yield), mp 93-95° C. MS (FAB): m/z 454 (100%) [M+Na]+. 1H NMR (500 MHz, DMSO-d6) δ 8.91 (s, 1H), 7.8-7.3 (m, 9H), 4.45 (br s, 1H), 4.33 (d, J=11.8 Hz, 1H), 4.20 (m, 2H), 3.3-3.2 (m, 2H), 1.15 (d, J=6.7 Hz, 3H). 13C RMN (125 MHz, DMSO-d6) δ 159.9, 154.5, 153.9, 147.7, 143.9, 126.8, 126.8, 126.5, 125.6, 125.5, 123.8, 122.6, 119.4 111.8, 111.4, 54, 48.7, 46.8, 14.9. HRMS (m/z): calcd. for C₂₂H₂₀F₃N₃O₃Na 454.1349 [M+Na]+; found 454.1340. Anal. calcd C22H20F3N3O3: C, 61.25; H, 4.67; N, 9.74. Found: C, 61.49; H, 4.90; N, 9.43.

4-(Benzofuran-2-carbonyl)-1-[(4-cyanophenyl)aminocarbonyl]-2-methylpiperazine (34). The product was obtained as a solid and purified by column chromatography using hexane-ethyl acetate (1:2) as eluent (367 mg, 95% yield), mp 97-99° C. MS (FAB): m/z 411 (100%) [M+Na]+. 1H NMR (500 MHz, DMSO-d6) δ 9.02 (s, 1H), 7.7-7.3 (m, 9H), 4.42 (br s, 1H), 4.30 (d, J=11.7 Hz, 1H), 4.10 (d, J=12.0 Hz, 2H), 3.23 (t, J=11.5 Hz, 2H), 1.12 (d, J=7.2 Hz, 3H). 13C RMN (125 MHz, DMSO-d6) δ 159.9, 154.2, 153.9, 144.8, 132.8, 126.8, 126.5, 125.6, 123.8, 122.6, 119.4, 118.6, 113.6, 111.7, 111.4, 103.3, 59.9, 46.8, 15.0. HRMS (m/z): calcd. for C22H20N4O3Na 411.1428 [M+Na]+; found 411.1418. Anal. calcd C22H20N4O3: C, 68.03; H, 5.19; N, 14.42. Found: C, 68.17; H, 5.10; N, 14.75.

4-(Benzofuran-2-carbonyl)-1-[(4-fluorophenyl)aminocarbonyl]-2-methylpiperazine (35). The product was obtained as a solid and purified by column chromatography using hexane-ethyl acetate (2:1) as eluent (320 mg, 84% yield), mp 93-95° C. MS (FAB): m/z 404 (100%) [M+Na]+. 1H NMR (500 MHz, DMSO-d6) δ 8.59 (s, 1H), 7.78-7.05 (m, 9H), 4.42 (br s, 2H), 4.33-4.27 (m, 2H), 4.23-4.13 (m, 1H), 3.24-3.19 (m, 2H), 1.14 (d, J=6.6 Hz, 3H). 13C RMN (125 MHz, DMSO-d6) δ 158.6, 155.1, 153.9, 147.7, 136.15, 136.13, 126.8, 126.5, 123.8, 122.6, 122.2, 122.1, 114.9, 114.7, 46.7, 26.7, 14.9. HRMS (m/z): calcd. for C21H20FN3O3Na 404.1381 [M+Na]+; found 404.1369. Anal. calcd C21H20FN3O3: C, 66.13; H, 5.29; N, 11.02. Found: C, 66.07; H, 5.59; N, 11.19.

4-(Benzofuran-2-carbonyl)-2-methyl-1-[(2-nitrophenyl)aminocarbonyl]piperazine (36). The product was obtained as a solid and purified by column chromatography using hexane-ethyl acetate (2:1) as eluent (294 mg, 95% yield), mp 141-143° C. MS (FAB): m/z 431 (100%) [M+Na]+. 1H NMR (500 MHz, DMSO-d6) δ 9.37 (s, 1H), 7.94-7.22 (m, 9H), 4.39-4.29 (m, 2H), 4.22-4.14 (d, 2H), 3.51-3.39 (m, 1H), 3.35-3.20 (m, 2H), 1.19 (d, J=6.6 Hz, 3H). 13C RMN (125 MHz, DMSO-d6) δ 159.9, 154.3, 153.9, 147.6, 140.8, 134.14, 134.11, 126.8, 126.5, 125.0, 123.9, 123.8, 123.4, 122.6, 111.7, 111.5, 47.3, 15.1. HRMS (m/z): calcd. for C21H20N4O5Na 431.1326 [M+Na]+; found 431.1319. Anal. calcd C21H20N4O5: C, 61.76; H, 4.94; N, 13.72. Found: C, 62.05; H, 5.07; N, 13.41.

4-(Benzofuran-2-carbonyl)-1-[(2-fluorophenyl)aminocarbonyl]-2-methylpiperazine (37). The product was obtained as a solid and purified by column chromatography using hexane-ethyl acetate (2:1) as eluent (267 mg, 70% yield), mp 127-129° C. MS (FAB): m/z 404 (100%) [M+Na]+. 1H NMR (500 MHz, DMSO-d6) δ 8.38 (s, 1H), 7.78-7.10 (m, 9H), 4.40 (br s, 1H), 4.34-4.32 (m, 1H), 4.18-4.11 (m, 2H), 3.25-3.21 (m, 2H), 1.15 (d, J=6.6 Hz, 3H). 13C RMN (125 MHz, DMSO-d6) δ 159.9, 155.2, 154.0, 147.7, 127.0, 126.8, 126.5, 125.6, 125.5, 124.0, 123.8, 122.6, 115.6, 111.7, 47.0, 14.8. HRMS (m/z): calcd. for C21H20FN3O3Na 404.1381 [M++Na]+; found 404.1372. Anal. calcd C21H20FN3O3: C, 66.13; H, 5.29; N, 11.02. Found: C, 66.25; H, 5.60; N, 10.85.

4-(Benzofuran-2-carbonyl)-1-[(2-bromophenyl)aminocarbonyl]-2-methylpiperazine (38). The product was obtained as a solid and purified by column chromatography using hexane-ethyl acetate (1.5:1) as eluent (380 mg, 86% yield), mp 129-131° C. MS (FAB): m/z 464 (100%) [M+Na]+. 1H NMR (500 MHz, DMSO-d6) δ 8.25 (s, 1H), 7.78-7.08 (m, 9H), 4.40 (br s, 1H), 4.33-4.32 (m, 1H), 4.20-4.17 (m, 1H), 3.27-3.22 (m, 2H), 1.19 (d, J=6.6 Hz, 3H). 13C RMN (125 MHz, DMSO-d6) δ 159.9, 155.1, 153.9, 147.7, 137.5, 132.4, 128.0, 127.6, 126.8, 126.6, 123.8, 122.6, 119.8, 111.7, 78.8, 47.1, 15.0. HRMS (m/z): calcd. for C21H20BrN3O3Na 464.0580 [M+Na]+; found 464.0574. Anal. calcd C21H20BrN3O3: C, 57.02; H, 4.56; N, 9.50. Found: C, 57.16; H, 4.49; N, 9.40.

4-(Benzofuran-2-carbonyl)-1-[(2,4-difluorophenyl)aminocarbonyl]-2-methylpiperazine (39). The product was obtained as a solid and purified by column chromatography using hexane-ethyl acetate (1:1) as eluent (323 mg, 81% yield), mp 163-165° C. MS (FAB): m/z 422 (100%) [M+H]+. 1H NMR (500 MHz, DMSO-d6) δ 8.40 (s, 1H), 7.78-7.00 (m, 8H), 4.38 (br s, 1H), 4.33-4.31 (m, 1H), 4.20-4.18 (m, 1H), 3.24-3.20 (m, 2H), 1.15 (d, J=6.6 Hz, 3H). 13C RMN (125 MHz, DMSO-d6) δ 159.9, 155.3, 154.0, 147.7, 128.1, 128.0, 126.8, 126.5, 123.8, 122.6, 111.7, 111.4, 47.0, 14.8. HRMS (m/z): calcd. for C21H19F2N3O3Na 422.1287 [M+Na]+; found 422.1275. Anal. calcd C21H19F2N3O3: C, 63.15; H, 4.80; N, 10.52. Found: C, 63.35; H, 4.97; N, 10.06.

4-(Benzofuran-2-carbonyl)-1-[(2-methoxyphenyl)aminocarbonyl]-2-methylpiperazine (40). The product was obtained as a solid and purified by column chromatography using hexane-ethyl acetate (1:1) as eluent (373 mg, 95% yield), mp 127-129° C. MS (FAB): m/z 416 (100%) [M+Na]+. 1H NMR (500 MHz, DMSO-d6) δ 8.15 (s, 1H), 7.78-7.00 (m, 9H), 4.38 (br s, 1H), 4.32-4.30 (m, 1H), 4.20-4.15 (m, 2H), 3.79 (s, 3H), 3.26-3.22 (m, 2H), 1.15 (d, J=6.6 Hz, 3H). 13C RMN (125 MHz, DMSO-d6) δ 160.0, 155.0, 154.0, 147.7, 128.0, 126.8, 126.5, 123.8, 122.7, 120.2, 111.8, 111.0, 55.7, 47.0, 14.9. HRMS (m/z): calcd. for C22H23N3O4Na 416.1581 [M+Na]+; found 416.1568. Anal. calcd C22H23N3O4: C, 67.16; H, 5.89; N, 10.68. Found: C, 67.34; H, 6.08; N, 10.49.

4-(Benzofuran-2-carbonyl)-2-methyl-1-(phenylaminocarbonyl)piperazine (41). The product was obtained as a solid and purified by column chromatography using hexane-ethyl acetate (1.5:1) as eluent (352 mg, 97% yield), mp 138-140° C. MS (FAB): m/z 386 (100%) [M+Na]+. 1H NMR (500 MHz, DMSO-d6) δ 8.54 (s, 1H), 7.78-7.00 (m, 10H), 4.44 (br s, 1H), 4.34-4.31 (m, 1H), 4.25-4.07 (m, 2H), 3.24-3.17 (m, 2H), 1.14 (d, J=6.5 Hz, 3H). 13C RMN (125 MHz, DMSO-d6) δ 155.2, 154.0, 140.0, 128.4, 126.8, 126.5, 123.8, 122.6, 122.3, 120.2, 111.8, 111.4, 78.8, 46.7, 14.9. HRMS (m/z): calcd. for C21H21N3O3Na 386.1475 [M+Na]+; found 386.1463. Anal. calcd C21H21N3O3: C, 69.41; H, 5.82; N, 11.56. Found: C, 69.62; H, 5.95; N, 11.33.

4-tert-Butoxycarbonyl-1-[(4-nitrophenyl)aminocarbonyl]-2-phenylpiperazine (46). The product was obtained as a solid and purified by column chromatography using hexane-ethyl acetate (1.5:1) as eluent (213 mg, 50% yield), mp 101-103° C. MS (FAB): m/z 449 (100%) [M+Na]+. 1H NMR (500 MHz, CDCl3) δ 8.0-7.3 (m, 9H), 6.65 (m, 1H), 5.1 (m, 1H), 4.5-3.3 (m 6H), 1.41 (s, 9H). 13C RMN (125 MHz, CDCl3) δ 154.3, 144.9, 142.7, 138.3, 129.7, 126.4, 125.0, 118.4, 113.4, 80.5, 58.6, 49.5, 46.6, 43.1, 28.3. HRMS (m/z): calcd. for C22H26N4O5Na 449.1796 [M+Na]+; found 449.1781. Anal. calcd C22H26N4O05: C, 61.96; H, 6.15; N, 13.14. Found: C, 62.15; H, 6.19; N, 12.91.

4-tert-Butoxycarbonyl-1-[(4-chlorophenyl)aminocarbonyl]-2-phenylpiperazine (47). The product was obtained as a solid and purified by column chromatography using hexane-ethyl acetate (5:1) as eluent (407 mg, 98% yield), mp 115-117° C. MS (FAB): m/z 438 (100%) [M+Na]+. 1H NMR (500 MHz, DMSO-d6) δ 8.77 (s, 1H), 7.53-7.50 (m, 2H), 7.39-7.35 (m, 2H), 7.32-7.26 (m, 5H), 5.47 (s, 1H), 4.45 (br s, 1H), 4.02 (m 1H), 3.89-3.57 (m 1H), 3.15-3.03 (m 2H), 1.35 (s, 9H). 13C RMN (125 MHz, DMSO-d6) δ 154.9, 153.5, 139,4, 131.6, 128.4, 128.2, 126.9, 126.4, 125.5, 121.2, 79.1, 53.1, 48.7, 46.0, 42.3, 27.9. HRMS (m/z): calcd. for C22H26C1N3O3Na 438.1555 [M+Na]+; found 438.1542. Anal. calcd C22H26C1N3O3: C, 63.53; H, 6.30; N, 10.10. Found: C, 63.17; H, 6.20; N, 9.73.

4-tert-Butoxycarbonyl-2-phenyl-1-[(4-trifluoromethylphenyl)aminocarbonyl]piperazine (48). The product was obtained as a syrup and purified by column chromatography using hexane-ethyl acetate (3:1) as eluent (202 mg, 45% yield). MS (FAB): m/z 472 (100%) [M+Na]+. 1H NMR (500 MHz, CDCl3) δ 7.5-7.2 (m, 9H), 6.50 (s, 1H), 5.1 (m, 1H), 4.5-3.1 (m 6H), 1.40 (s, 9H). 13C RMN (125 MHz, CDCl3) δ 154.8, 154.4, 142.0, 138.4, 129.6, 128.6, 123.1, 119.1, 114.2, 80.4, 58.1, 46.5, 44.1, 39.4, 28.4. HRMS (m/z): calcd. for C23H26F3N3O3Na 472.1818 [M+Na]+; found 472.1803.

4-tert-Butoxycarbonyl-1-[(4-fluorophenyl)aminocarbonyl]-2-phenylpiperazine (49). The product was obtained as a solid and purified by column chromatography using hexane-ethyl acetate (3:1) as eluent (315 mg, 79% yield), mp 202-204° C. MS (FAB): m/z 422 (100%) [M+Na]+. 1H NMR (500 MHz, DMSO-d6) δ 8.68 (s, 1H), 7.5-7.1 (m, 9H), 5.47 (s, 1H), 4.45 (br s, 1H), 4.05-3.97 (m 1H), 3.87-3.71 (m 1H), 3.14-2.93 (m 2H), 1.35 (s, 9H). 13C RMN (125 MHz, DMSO-d6) δ 158.4, 156.5, 139.1, 136.6, 128.4, 126.6, 121.6, 121.5, 114,9, 114.7, 79.1, 53.1, 46.1, 27.9. HRMS (m/z): calcd. for C22H26FN3O3Na 422.1850 [M+Na]+; found 422.1838. Anal. calcd C22H26FN3O3: C, 66.15; H, 6.56; N, 10.52. Found: C, 66.21; H, 6.46; N, 10.22.

4-tert-Butoxycarbonyl-1-[(4-cyanophenyl)aminocarbonyl]-2-phenylpiperazine (50). The product was obtained as a solid and purified by column chromatography using hexane-ethyl acetate (1.5:1) as eluent (390 mg, 96% yield), mp 94-96° C. MS (FAB): m/z 429 (100%) [M+Na]+. 1H NMR (500 MHz, CDCl3) δ 7.5-7.2 (m, 9H), 6.55 (s, 1H), 5.10 (s, 1H), 4.5-3.2 (m, 6H), 1.40 (s, 9H). 13C RMN (125 MHz, CDCl3) δ 154.4, 142.7, 138.3, 133.1, 129.7, 126.2, 119.1, 114.5, 105.8, 80.5, 58.6, 49.5, 44.2, 38.5, 28.3. HRMS (m/z): calcd. for C23H26N4O3Na 429.1897 [M+Na]+; found 429.1897. Anal. calcd C23H26N4O3: C, 67.96; H, 6.45; N, 13.78. Found: C, 68.03; H, 6.37; N, 13.27.

4-tert-Butoxycarbonyl-1-[(2-nitrophenyl)aminocarbonyl]-2-phenylpiperazine (51). The product was obtained as a solid and purified by column chromatography using hexane-ethyl acetate (5:1) as eluent (384 mg, 90% yield), mp 132-134° C. MS (FAB): m/z 449 (100%) [M+Na]+. 1H NMR (500 MHz, DMSO-d6) δ 9.41 (s, 1H), 7.96 (dd, J=1.2 Hz, J=8.2 Hz, 1H), 7.75 (d, J=8.1 Hz, 1H), 7.67 (dt, J=1.2 Hz, J=7.2 Hz, J=8.3 Hz, 1H), 7.38 (m, 4H), 7.30 (t, J=7.2 Hz, 1H), 7.24 (td, J=1.2 Hz, J=7.0 Hz, J=8.1 Hz, 1H), 5.39 (s, 1H), 4.45 (br s, 1H), 4.00 (m 1H), 3.88-3.67 (m 1H), 3.47-3.40 (m 1H), 3.24-2.94 (m, 2H), 1.36 (s, 9H). 13C RMN (125 MHz, DMSO-d6) δ 154.5, 140.6, 134.4, 134.1, 128.4, 127.0, 126.6, 125.50, 123.9, 123.1, 79.1, 53.8, 46.1, 42.4, 27.9. HRMS (m/z): calcd. for C22H26N4O5Na 449.1795 [M+Na]+; found 449.1782. Anal. calcd C22H26N4O5: C, 61.96; H, 6.15; N, 13.14. Found: C, 61.98; H, 5.88; N, 12.97.

4-tert-Butoxycarbonyl-1-[(2-chloro-5-trifluoromethylphenyl)aminocarbonyl]-2-phenylpiperazine (52). The product was obtained as a solid and purified by column chromatography using hexane-ethyl acetate (9:1) as eluent (473 mg, 98% yield), mp 96-98° C. MS (FAB): m/z 506 (100%) [M+Na]+. 1H NMR (500 MHz, DMSO-d6) δ 8.39 (s, 1H), 8.03-7.31 (m, 8H), 5.41 (s, 1H), 4.44-4.20 (m, 1H), 4.05-3.99 (m 1H), 3.88-3.66 (m 1H), 3.56-3.47 (m, 1H), 3.31-3.23 (m 1H), 1.33 (s, 9H). 13C RMN (125 MHz, DMSO-d6) δ 154.7, 153.5, 138.9, 137.5, 130.5, 127.1, 126.6, 124.8, 122.6, 121.5, 79.1, 54.0, 46.2, 27.9. HRMS (m/z): calcd. for C23H25C1F3N3O3Na 506.1429 [M+Na]+; found 506.1417. Anal. calcd C23H25C1F3N3O3: C, 57.09; H, 5.21; N, 8.68. Found: C, 57.04; H, 5.34; N, 8.46.

4-tert-Butoxycarbonyl-1-[(4-chloro-3-trifluoromethylphenyl)aminocarbonyl]-2-phenylpiperazine (53). The product was obtained as a solid and purified by column chromatography using hexane-ethyl acetate (5:1) as eluent (464 mg, 96% yield), mp 109-111° C. MS (FAB): m/z 506 (100%) [M+Na]+. 1H NMR (500 MHz, DMSO-d6) δ 9.01 (s, 1H), 8.08 (d, J=2.5 Hz, 1H), 7.83 (dd, J=2.5 Hz, J=8.9 Hz, 1H), 7.60 (d, J=8.9 Hz, 1H), 7.41-7.35 (m, 2H), 7.32-7.27 (m, 3H), 5.48 (s, 1H), 4.47 (br s, 1H), 4.02 (m 1H), 3.89-3.63 (m 1H), 3.44-3.37 (m, 1H), 3.15-2.91 (m 2H), 1.35 (s, 9H). 13C RMN (125 MHz, DMSO-d6) δ 154.6, 153.6, 140.1, 138.8, 131.6, 128.4, 126.9, 124.0, 122.3, 121.8, 118.0, 79.1, 54.1, 45.9, 27.9. HRMS (m/z): calcd. for C23H25C1F3N3O3Na 506.1429 [M+Na]+; found 506.1418. Anal. calcd C23H25C1F3N3O3: C, 57.09; H, 5.21; N, 8.68. Found: C, 56.85; H, 5.27; N, 8.49.

4-tert-Butoxycarbonyl-1-[(4-methoxyphenyl)aminocarbonyl]-2-phenylpiperazine (54). The product was obtained as a solid and purified by column chromatography using hexane-ethyl acetate (2:1) as eluent (370 mg, 90% yield), mp 90-92° C. MS (FAB): m/z 434 (100%) [M+Na]+. 1H NMR (500 MHz, CDCl3) δ 7.4-7.3 (m, 9H), 6.08 (s, 1H), 5.08 (m, 1H), 4.3-3.4 (m 6H), 3.75 (s, 3H), 1.40 (s, 9H). 13C RMN (125 MHz, CDCl3) δ 156.0, 155.7, 138.9, 131.7, 129.4, 128.5, 126.5, 123.7, 122.2, 114.1, 80.2, 58.0, 55.5, 49.4, 45.2, 39.4, 28.3. HRMS (m/z): calcd. for C23H29N3O4Na 434.2050 [M+Na]+; found 434.2037. Anal. calcd C23H29N3O4: C, 67.13; H, 7.10; N, 10.21. Found: C, 67.32; H, 6.93; N, 9.98.

4-tert-Butoxycarbonyl-1-[(4-methylphenyl)aminocarbonyl]-2-phenylpiperazine (55). The product was obtained as a solid and purified by column chromatography using hexane-ethyl acetate (3:1) as eluent (348 mg, 88% yield), mp 162-164° C. MS (FAB): m/z 418 (100%) [M+Na]+. 1H NMR (500 MHz, CDCl3) δ 7.4-7.3 (m, 9H), 6.15 (s, 1H), 5.08 (m, 1H), 4.4-3.3 (m 6H), 2.25 (s, 3H), 1.33 (s, 9H). 13C RMN (125 MHz, CDCl3) δ 150.2, 133.6, 130.8, 127.6, 124.1, 123.2, 121.0, 114.9, 75.0, 52.8, 41.4, 39.0, 34.1, 23.1, 15.5. HRMS (m/z): calcd. for C23H29N3O3Na 418.2101 [M+Na]+; found 418.2088. Anal. calcd C23H29N3O3: C, 69.85; H, 7.39; N, 10.62. Found: C, 70.03; H, 7.55; N, 10.42.

1-[(4-Nitrophenyl)aminocarbonyl]-2-phenyl-4-pivaloylpiperazine (56). The product was obtained as a solid and purified by column chromatography using hexane-ethyl acetate (1.5:1) as eluent (352 mg, 97% yield). MS (FAB): m/z 433 (65%) [M+Na]+. 1H NMR (500 MHz, DMSO-d6) δ 9.31 (s, 1H), 8.22-7.23 (m, 9H), 5.42 (m, 1H), 1.10 (s, 9H). 13C RMN (125 MHz, DMSO-d6) δ 175.4, 154.2, 155.3, 140.1, 132.5, 128.6, 125.6, 122.2, 120.5, 113.8, 113.5, 79.5, 55.4, 45.4, 38.1, 27.7. HRMS (m/z): calcd. for C22H26N4O4Na 433.1852 [M+Na]+; found 433.1833. Anal. calcd C22H26N4O4: C, 64.37; H, 6.38; N, 13.65. Found: C, 64.58; H, 6.19; N, 13.51.

1-[(4-Cyanophenyl)aminocarbonyl]-2-phenyl-4-pivaloylpiperazine (57). The product was obtained as a solid and purified by column chromatography using hexane-ethyl acetate (1:1) as eluent (261 mg, 67% yield), mp 151-153° C. MS (FAB): m/z 413 (100%) [M+Na]+. 1H NMR (500 MHz, DMSO-d6) δ 8.42 (s, 1H), 7.3-6.8 (m, 9H), 5.42 (m, 1H), 4.56 (d, J=12.9 Hz, 1H), 3.98 (td, J=3.4 Hz, J=13.0 Hz, 1H), 3.98 (d, J=13.0 Hz, 1H), 3.45 (d, J=11.2 Hz, 1H), 3.38 (m, 1H), 3.29 (m, 1H), 3.75 (s, 3H), 1.08 (s, 9H). 13C RMN (125 MHz, DMSO-d6) δ 176.0, 154.7, 139.7, 132.7, 128.3, 126.3, 121.9, 120.2, 113.8, 113.5, 79.0, 55.2, 55.1, 45.4, 38.1, 27.7. HRMS (m/z): calcd. for C23H26N4O2Na 413.1948 [M+Na]+; found 413.1933. Anal. calcd C23H26N4O2: C, 70.75; H, 6.71; N, 14.35. Found: C, 70.87; H, 6.46; N, 14.64.

1-[(4-Methoxyphenyl)aminocarbonyl]-2-phenyl-4-pivaloylpiperazine (58). The product was obtained as a solid and purified by column chromatography using hexane-ethyl acetate (1:1) as eluent (229 mg, 58% yield), mp 62-65° C. MS (FAB): m/z 418 (55%) [M+Na]+. 1H NMR (500 MHz, DMSO-d6) δ 8.42 (s, 1H), 7.3-6.8 (m, 9H), 5.42 (m, 1H), 4.56 (d, J=12.9 Hz, 1H), 3.98 (td, J=3.4 Hz, J=13.0 Hz, 1H), 3.98 (d, J=13.0 Hz, 1H), 3.45 (d, J=11.2 Hz, 1H), 3.38 (m, 1H), 3.29 (m, 1H), 3.75 (s, 3H), 1.08 (s, 9H). 13C RMN (125 MHz, DMSO-d6) δ 176.0, 154.7, 139.7, 132.7, 128.3, 126.3, 121.9, 120.2, 113.8, 113.5, 79.0, 55.2, 55.1, 45.4, 38.1, 27.7. HRMS (m/z): calcd. for C23H29N3O3Na 418.2101 [M+Na]+; found 418.2088. Anal. calcd C23H29N3O3: C, 69.85; H, 7.39; N, 10.62. Found: C, 69.95; H, 7.55; N, 10.37.

4-(Benzofuran-2-carbonyl)-1-[(4-nitrophenyl)aminocarbonyl]-2-phenylpiperazine (59). The product was obtained as a solid and purified by column chromatography using hexane-ethyl acetate (2:1) as eluent (400 mg, 85% yield), mp 141-143° C. MS (FAB): m/z 493 (100%) [M+Na]+. 1H NMR (500 MHz, DMSO-d6) δ 9.39 (s, 1H), 8.16 (m, 2H), 7.76-7.71 (m, 3H), 7.67-7.64 (m, 1H), 7.46 (m, 1H), 7.39-7.20 (m, 7H), 5.62 (br s, 1H), 4.80 (br s, 1H), 4.18 (m, 2H), 3.76-3.42 (m, 2H). 13C RMN (125 MHz, DMSO-d6) δ 159.3, 154.3, 153.9, 147.9, 147.2, 145.7, 141.5, 14.1, 138.9, 128.5, 127.1, 126.7, 126.3, 125.1, 124.7, 123.7, 122.5, 118.6, 111.8, 54.1, 48.7. HRMS (m/z): calcd. for C26H22N4O5Na 493.1482 [M+Na]+; found 493.1470. Anal. calcd C26H22N4O5: C, 66.37; H, 4.71; N, 11.91. Found: C, 66.58; H, 4.93; N, 11.71.

4-(Benzofuran-2-carbonyl)-1-[(4-chlorophenyl)aminocarbonyl]-2-phenylpiperazine (60). The product was obtained as a solid and purified by column chromatography using hexane-ethyl acetate (2:1) as eluent (414 mg, 90% yield), mp 118-120° C. MS (FAB): m/z 482 (100%) [M+Na]+. 1H NMR (500 MHz, DMSO-d6) δ 8.82 (s, 1H), 7.76-7.26 (m, 14H), 5.59 (br s, 1H), 4.81 (br s, 1H), 4.15 (m, 2H), 3.80-3.40 (m, 2H). 13C RMN (125 MHz, DMSO-d6) δ 159.3, 158.4, 153.9, 147.9, 139.3, 128.5, 128.2, 127.0, 126.7, 126.3, 125.6, 123.7, 122.5, 121.2, 111.8, 53.5. HRMS (m/z): calcd. for C26H22C1N3O3Na 482.1242 [M+Na]+; found 482.1237. Anal. calcd C26H22C1N3O3: C, 67.90; H, 4.82; N, 9.14. Found: C, 68.15; H, 5.01; N, 8.93.

4-(Benzofuran-2-carbonyl)-2-phenyl-1-[(4-trifluoromethylphenyl)aminocarbonyl]piperazine (61). The product was obtained as a solid and purified by column chromatography using hexane-ethyl acetate (2:1) as eluent (468 mg, 95% yield), mp 134-136° C. MS (FAB): m/z 516 (100%) [M+Na]+. 1H NMR (500 MHz, DMSO-d6) δ 9.10 (s, 1H), 7.77-7.26 (m, 14H), 5.63 (br s, 1H), 4.81 (br s, 1H), 4.18 (m, 2H), 3.79-3.49 (m, 2H). 13C RMN (125 MHz, DMSO-d6) δ 159.3, 154.7, 153.9, 147.9, 144.1, 139.0, 128.5, 127.1, 126.7, 126.3, 125.6, 123.7, 122.5, 119.1, 111.8, 53.9, 48.7. HRMS (m/z): calcd. for C27H22F3N3O3Na 516.1505 [M+Na]+; found 516.1494. Anal. calcd C27H22F3N3O3: C, 65.72; H, 4.49; N, 8.52. Found: C, 65.51; H, 4.18; N, 8.82.

4-(Benzofuran-2-carbonyl)-1-[(4-fluorophenyl)aminocarbonyl]-2-phenylpiperazine (62). The product was obtained as a solid and purified by column chromatography using hexane-ethyl acetate (2:1) as eluent (337mg, 76% yield), mp 122-124° C. MS (FAB): m/z 466 (100%) [M+Na]+. 1H NMR (500 MHz, DMSO-d6) δ 8.74 (s, 1H), 7.78-7.10 (m, 14H), 5.61 (br s, 1H), 4.81 (br s, 1H), 4.18 (m, 2H), 3.78-3.48 (m, 2H). 13C RMN (125 MHz, DMSO-d6) δ 159.3, 158.4, 156.6, 155.1, 153.9, 147.9, 139.2, 136.6, 128.5, 127.0, 126.6, 126.3, 123.7, 122.5, 121.6, 114.9, 111.7, 53.7, 48.7. HRMS (m/z): calcd. for C26H22FN3O3Na 466.1537 [M+Na]+; found 466.1524. Anal. calcd C26H22FN3O3: C, 70.42; H, 5.00; N, 9.48. Found: C, 70.22; H, 5.38; N, 9.19.

4-(Benzofuran-2-carbonyl)-1-[(4-cyanophenyl)aminocarbonyl]-2-phenylpiperazine (63). The product was obtained as a solid and purified by column chromatography using hexane-ethyl acetate (1:1) as eluent (437 mg, 97% yield), mp 212-214° C. MS (FAB): m/z 473 (100%) [M+Na]+. 1H NMR (500 MHz, DMSO-d6) δ 9.19 (s, 1H), 7.78-7.63 (m, 6H), 7.49-7.22 (m, 8H), 7.67-7.64 (m, 1H), 7.46 (m, 1H), 7.39-7.20 (m, 7H), 5.62 (br s, 1H), 4.81 (br s, 1H), 4.18 (m, 2H), 3.77-3.44 (m, 2H). 13C RMN (125 MHz, DMSO-d6) δ 159.3, 154.4, 153.9, 147.9, 144.9, 139.0, 132.9, 128.5, 127.1, 126.7, 126.3, 125.1, 124.7, 123.7, 122.5, 119.2, 118.4, 111.8, 103.3, 53.8, 40.7. HRMS (m/z): calcd. for C27H22N4O3Na 473.1584 [M+Na]+; found 473.1569. Anal. calcd C27H22N4O3: C, 71.99; H, 4.92; N, 12.44. Found: C, 71.88; H, 5.15; N, 12.21.

4-(Benzofuran-2-carbonyl)-1-[(2-nitrophenyl)aminocarbonyl]-2-phenylpiperazine (64). The product was obtained as a solid and purified by column chromatography using hexane-ethyl acetate (2:1) as eluent (348 mg, 74% yield), mp 143-145° C. MS (FAB): m/z 493 (100%) [M+Na]+. 1H NMR (500 MHz, DMSO-d6) 6 9.45 (s, 1H), 7.98 (dd, J=1.2 Hz, J=8.4 Hz, 1H), 7.8 (d, J=8.4 Hz, 1H), 7.76 (d, J=7.7 Hz, 1H), 7.69-7.66 (m 2H), 7.50-7.23 (m, 9H), 5.23 (br s, 1H), 4.77 (br s, 1H), 4.15 (m, 2H), 3.84-3.41 (m, 2H). 13C RMN (125 MHz, DMSO-d6) δ 159.3, 154.4, 153.9, 147.9, 138.6, 134.4, 134.2, 128.5, 127.2, 126.7, 126.3, 125.1, 123.7, 123.1, 122.5, 111.7, 48.7. HRMS (m/z): calcd. for C26H22N4O5Na 493.1482 [M+Na]+; found 493.1470. Anal. calcd C26H22N4O5: C, 66.37; H, 4.71; N, 11.91. Found: C, 66.27; H, 4.83; N, 11.73.

4-(Benzofuran-2-carbonyl)-1-[(2-chloro-5-trifluoromethylphenyl)aminocarbonyl]-2 phenylpiperazine (65). The product was obtained as a solid and purified by column chromatography using hexane-ethyl acetate (2.5:1) as eluent (486 mg, 92% yield), mp 140-142° C. MS (FAB): m/z 550 (100%) [M+Na]+. 1H NMR (500 MHz, DMSO-d6) δ 8.07 (s, 1H), 7.75 (d, J=8.4 Hz, 1H), 7.70-7.66 (m, 2H), 7.49-7.44 (m, 3H), 7.40-7.29 (m, 7H), 5.53(br s, 1H), 4.66 (br s, 1H), 4.18 (m, 2H), 3.89-3.48 (m, 2H). 13C RMN (125 MHz, DMSO-d6) δ 159.4, 154.5, 153.9, 147.9, 138.9, 137.4, 130.4, 128.6, 127.4, 126.7, 126.3, 124.8, 123.7, 122.5, 121.4, 111.8, 48.7. HRMS (m/z): calcd. for C27H21C1F3N3O3Na 550.1116 [M+Na]+; found 550.1104. Anal. calcd C27H21C1F3N3O3: C, 61.43; H, 4.01; N, 7.96. Found: C, 61.00; H, 4.20; N, 7.73.

1.2. Biological evaluation. Cells and virus. Human A549, 293 and MRC-5 cell lines were from the American Type Culture Collection (ATCC, Manassas, Va.). The 293β5 stable cell line overexpressing the human β5 integrin subunit was generated by transfecting a cytomegalovirus promoter-driven expression plasmid containing the human β5 gene into 293 cells and selecting for neomycin resistance.40 These cell lines were propagated in Dulbecco's modified Eagle medium (DMEM, Life Technologies/Thermo Fisher) supplemented with 10% fetal bovine serum (FBS) (Omega Scientific, Tarzana, Calif.), 10 mM HEPES, 4 mM L-glutamine, 100 units/ml penicillin, 100 μg/ml streptomycin, and 0.1 mM non-essential amino acids (complete DMEM).

Wild-type HAdV5 and HAdV16 and HCMV (AD169) were obtained from ATCC. The HAdV5-GFP and HAdV16-GFP used in this work are replication-defective viruses containing a CMV promoter-driven enhanced green fluorescent protein (eGFP) reporter gene cassette in place of the E1/E3 regions.41 HAdV viruses were propagated in 293β5 cells and isolated from cellular lysate by cesium chloride density centrifugation. Virus concentration, in mg/ml, was calculated with the Bio-Rad Protein Assay (Bio-Rad Laboratories) and converted to virus particles/ml (vp/ml) using 4×1012 vp/mg.

1.3. Entry assay. An initial rapid screening was performed using human A549 epithelial cells (3×105 cells/well in Corning black wall, clear bottom 96-well plates) infected with HAdV5-GFP (2,000 vp/cell) in the presence of 50 μM of the candidate antiviral compounds. Virus was preincubated with the compounds (individual and mixed) for 45 minutes at 4° C., and then added to cells. A standard infection curve was generated in parallel by infecting cells in the absence of compounds using serial two-fold dilutions of virus. All reactions were done in triplicate. Cells, virus and compounds were incubated for 48 h at 37° C. and 5% CO2. Infection, as measured by HAdV-mediated GFP expression, was analyzed using a Typhoon 9410 imager (GE Healthcare Life Sciences), and quantified with ImageQuantTL (GE Healthcare Life Sciences). Compounds that showed antiviral activity were further tested in a dosage assay using 2,000 vp/cell and compound concentrations ranging 50 to 1.56 μM.

1.4. Cytotoxicity assay. The cytotoxicity of the compounds was measured using the AlarmBlue cell viability assay (Invitrogen) according to the manufacturer's instructions. Actively dividing A549 cells were incubated with compounds for 48 h. After the incubation the alamarBlue reagent was added to the cells ( 1/10th alamarBlue reagent in culture medium) for an extra 4 h. The 50% cell cytotoxic concentration (CC50) of the molecules was calculated according to Cheng et al.42 The selectivity index (SI) was evaluated as the ratio of CC50 to IC50, where the IC50 is defined as the concentration of compound that inhibits HAdV infection by 50%.

1.5. Plaque assay. For low MOI infections, active compounds were further evaluated in a plaque assay. 293β5 cells were seeded in 6-well plates at 4×105 cells per well in duplicate for each condition. When cells reached 80-90% confluency, they were infected with HAdV5-GFP or HAdV16-GFP (0.06 vp/cell) and rocked for 2 h at 37° C. The inoculum was removed and the cells were washed once with PBS. The cells were then carefully overlaid with 4 ml/well of equal parts of 1.6% (water/vol) Difco Agar Noble (Becton, Dickinson & Co, Sparks, MD) and 2× EMEM (BioWhittaker) supplemented with 2x penicillin/streptomycin, 2× L-glutamine and 10% FBS. The mixture also contained compound in concentrations ranging from 5 to 1 μM. Following incubation for 7 days at 37° C., plates were scanned with a Typhoon 9410 imager (GE Healthcare Life Sciences), and plaques were quantified with ImageJ.43

1.6. DNA quantification by real-time PCR. For DNA quantification, A549 cells (150,000 cells/well in a 24 wells-plate) were infected with wild type HAdV5 or HAdV16 (100 vp/cell) and incubated for 2 h at 37° C. in complete DMEM. After the incubation, excess virus was removed and the medium was replaced with 500 μl of complete DMEM containing 50 μM of either compounds or the same volume of DMSO (positive control). All samples were done in triplicate. After 24 h of incubation at 37° C., DNA was purified from the cell lysate with the QlAamp DNA Mini Kit (QIAGEN, Valencia, Calif.) following the manufacturer's instructions. TaqMan primers and probes for a region of the HAdV5 hexon were designed with the GenScript Real-time PCR (TaqMan) Primer Design software (GenScript). Oligonucleotides sequences were AdF: 5′-GACATGACTTTTGAGGTGGA-3′; AdR: 5′-GTGGCGTTGCCGGCCGAGAA-3′; and AdProbe: 5′-TCCATGGGATCCACCTCAAA-3′. Real-time PCR mixtures consisted of 2 μl the purified DNA, AdF and AdR at a concentration of 200 nM each, and AdProbe at a concentration of 50 nM in a total volume of 12.5 μl and mixed with 12.5 μl of KAPA PROBE FAST qPCR Master Mix (KAPABiosystems, MA). The PCR cycling protocol was 95° C. for 3 min followed by 40 cycles of 95° C. for 10 sec and 60° C. for 30 sec.

Human glyceraldehyde-3-phosphate dehydrogenase (GAPDH) gene was used as internal control. Oligonucleotides sequences for GAPDH and conditions were those previously reported by Rivera et al.44 For quantification, gene fragments from hexon and GAPDH were cloned into the pGEM-T Easy vector (Promega) and known concentrations of template were used to generate a standard curve in parallel for each experiment. All assays were performed in a C1000 Thermal Cycler apparatus (BioRad).

1.7. Nuclear-associated HAdV genomes. Nuclear delivery of the HAdV genome was assessed with real-time PCR following nuclear isolation from infected cells using a previously described protocol with a few modifications.45 Briefly, 1×106 A549 cells in 6-well plates were infected with HAdV5 wild type at MOI 2000 vp/cell in the presence of 50 μM of compound or the same volume of DMSO. Forty-five minutes after the infection, cytoplasmic and nuclear fractions were separated using a hypotonic buffer solution and NP-40 detergent. Following infection, A549 cells were trypsinized and collected, and then washed twice with PBS. The cell pellet was resuspended in 500 μl of 1× hypotonic buffer (20 mM Tris-HCl pH 7.4, 10 mM NaCl, 3 mM MgCl2) and incubated for 15 min at 4° C. Then, 25 μl of NP-40 was added and the samples were vortexed. The homogenates were centrifuged for 10 min at 835×g at 4° C. Following removal of the cytoplasmic fraction (supernatant), DNA was isolated from the nuclear fraction (pellet) using the QlAamp DNA Mini Kit (QIAGEN, Valencia, Calif.).

1.8. Virus yield reduction. The effect of the active compounds on virus production was evaluated in a burst assay A549 cells were infected with wild-type HAdV5 or HAdV16 (MOI 100) in the presence or absence of 50 μM compounds. After 48 h, cells were harvested and subjected to three rounds of freeze/thaw. Serial dilutions of clarified lysates were titrated on A549 cells and TCID50 values were calculated using an endpoint dilution method.46

1.9. HCMV infectivity assay by quantitative PCR. To test the sensitivity of HCMV to our compounds, MRC-5 cells (1.75×105 cells/well in a 6-well plate) were infected with HCMV at an MOI of 0.05 vp/cell and incubated in complete DMEM supplemented with 50 μM of compound or the same volume of DMSO in triplicate. After 72 h of incubation at 37° C., DNA was purified from the cell lysate with the QlAamp DNA Mini Kit (QIAGEN, Valencia, Calif.) following the manufacturer's instructions. TaqMan primers and probes for a region of the US28 gene were designed with the GenScript Real-time PCR (TaqMan) Primer Design software (GenScript). Oligonucleotides sequences were CMV-F: 5′-TCTACGTGGCTATGTTTGCC-3′; CMV-R: 5′-GGCCGATATCTCATGTAAACAA-3′; and CMV-Probe: 5′-CACGGAGATTGCACTCGATCGC-3′. Real-time PCR mixtures consisted of 10 μl the purified DNA, CMV-F at a concentration of 100 nM, CMV-R at a concentration of 300 nM, and CMV-Probe at a concentration of 50 nM in a total volume of 12.5 μl and mixed with 12.5 μl of KAPA PROBE FAST qPCR Master Mix (KAPABiosystems, MA). The PCR cycling protocol was 95° C. for 10 min followed by 40 cycles of 95° C. for 30 sec and 58° C. for 60 sec. Human glyceraldehyde-3-phosphate dehydrogenase (GAPDH) was used as internal control. Oligonucleotides sequences for GAPDH and conditions were those previously reported by Rivera et al.44 For quantification, gene fragment from US28 and GAPDH were cloned into the pGEM-T Easy vector (Promega) and known concentrations of template were used to generate a standard curve in parallel for each experiment. All assays were performed in a C1000 Thermal Cycler apparatus (BioRad).

1.10. Statistical analyses. Statistical analyses were performed with the GraphPad Prism 5 suite. Unless otherwise indicated, data are presented as the mean of triplicate samples ±standard deviation (SD).

### Example 2

### First-Generation Compounds

1. Chemistry. First-Generation Compounds.

2-methylpiperazine 5 (Scheme 1) was employed as the precursor of a new class of compounds which we called first generation compunds. Through a chemoselective N-acylation reaction of 2-substitued piperazines the acyl function at the less hindered nitrogen was introduced and the urethane and amide derivatives 6-9, with different R1, were synthesized. A general synthetic route for these compounds is presented in Scheme 1.

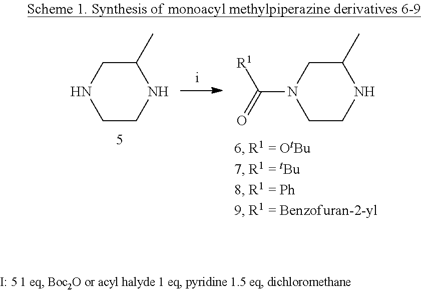

From these monoacylderivatives 6-9, the carbonyl group at the other nitrogen was incorporated as amide or urea/thiourea groups (Scheme 2) in order to determine which organic function at this position was critical for the biological activity. As our structure is now different from the models 2-4, the nature of the substituent on the phenyl ring (R2), electron withdrawing or electron-releasing group should be also evaluated. By choosing the appropriate acyl chloride or isocyanate/isothiocyanate reactants, following the route that is essentially described in Scheme 2, compounds 10-26 were obtained (Table 1).

Scheme 2. Synthetic methodology for the preparation of the new methylpiperazine derivatives

TABLE 1
First generation products.

| | 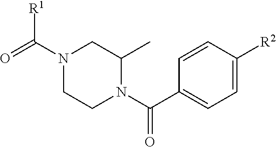 | | 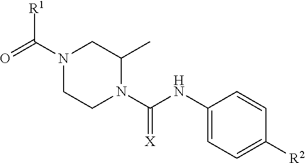 | |
|---|---|---|---|---|
| | A | | B | |
| Compound | Structure | R¹ | X | R² |
| 10 | A | O^{t}Bu | — | NO₂ |
| 11 | A | O^{t}Bu | — | OCH₃ |
| 12 | B | O^{t}Bu | S | NO₂ |
| 13 | B | O^{t}Bu | O | NO₂ |
| 14 | B | O^{t}Bu | O | OCH₃ |
| 15 | A | ^{t}Bu | — | NO₂ |
| 16 | A | ^{t}Bu | — | OCH₃ |
| 17 | B | ^{t}Bu | S | NO₂ |
| 18 | B | ^{t}Bu | O | NO₂ |
| 19 | B | ^{t}Bu | O | OCH₃ |
| 20 | A | Ph | — | NO₂ |
| 21 | A | Ph | — | OCH₃ |
| 22 | B | Ph | S | NO₂ |
| 23 | B | Ph | O | NO₂ |
| 24 | B | Ph | O | OCH₃ |
| 25 | B | Benzofuran-2-yl | O | NO₂ |
| 26 | B | Benzofuran-2-yl | O | OCH₃ |

2. Biology. First-Generation Compounds.

Compounds 10-26 were screened for their potential anti-HAdV activity by plaque assay, quantifying HAdV plaque formation in the presence of the candidate molecules and by entry assay, to evaluate the capacity of the candidate molecules to block HAdV entry into the cells.

For the plaque assay 293β5 cells were infected with HAdV5-GFP (in the presence of compound at 10 μM) (Table 2). From this generation of compounds, our primary screening identified 4 compounds (12, 13, 22 and 25) that inhibited HAdV5-GFP plaque formation >90% compared to a control with the same volume of DMSO.

TABLE 2
Inhibition of HAdV plaque formation for compounds 10-26

| | Percentage of plaque formation |
|---|---|
| Compound | inhibition^{a} |
| 10 | 33.65 ± 2.17 |
| 11 | 33.77 ± 4.02 |
| 12 | 100 ± 0 |
| 13 | 91.35 ± 3.27 |
| 14 | 49.13 ± 1.39 |
| 15 | 23.08 ± 3.71 |
| 16 | 0 |
| 17 | 64.04 ± 8.97 |
| 18 | 0 |
| 19 | 0 |
| 20 | 0 |
| 21 | 0 |
| 22 | 94.30 ± 2.82 |
| 23 | 71.66 ± 3.43 |
| 24 | 0 |
| 25 | 94.33 ± 1.56 |
| 26 | 33.60 ± 14.69 |

^{a}Percentage inhibition of HadV5-GFP in a plaque assay at 10 μM using the 293β5 cell line. The results represent means ± SD of triplicate samples from three independent experiments. See the Experimental Section for details.

From this generation of compounds, our primary screen identified 4 compounds (12, 13, 22 and 25) that inhibited HAdV5-GFP infection >90% in the plaque assay. For the entry assay, human A549 epithelial cells were infected with HAdV5-GFP in the presence of 50 μM of the candidate antiviral compound and incubated for 48 h. The obtained percentages of inhibition were not excessively high, for example 0% for 12 and 21, less than 20% for 10-12, 15-17, 19, 20 and 26 and only few compounds gave moderate-high (50-70%) values of inhibition (13, 18, 22-25). Table 3 shows the data found in the entry assay, as well as their evaluation for effects on cellular viability, for the most active compounds in the plaque assay.

TABLE 3
Percentage of control HAdV5-GFP infection and cytotoxicity
assay data for compounds 12, 13, 22 y 25.

| | Percentage of control | |
|---|---|---|
| Compound | HAdV5-GFP infection^{a} | CC₅₀ (μM)^{b} |
| 12 | 0 | 26.33 ± 1.59 |
| 13 | 64.55 ± 6.76 | 143.02 ± 3.29 |
| 22 | 41 ± 11.35 | 31.44 ± 2.82 |
| 25 | 86.22 ± 2.26 | 51.05 ± 7.35 |

^{a}Percentage inhibition to HAdV5-GFP in an entry assay at 50 μM using the A549 cell line.
^{b}Cytotoxic concentration 50.
The results represent means ± SD of triplicate samples from three independent experiments.

Results showed that among the molecules of this first generation the most active possessed the following structural features: they belong to general structure B (Table 1), containing urea or thiourea group at one nitrogen with an electron-withdrawing group, NO2 (R2, FIG. 1) and the acyl group on the other nitrogen is a urethane or a benzofuran-2-yl one (R1, FIG. 3).

As for cytotoxic activity, compounds showed low toxicity at concentrations higher than 100 μM. According to this, compounds 12 and 22 were cytotoxic (Table 3), so that thiourea function was finally discarded for the next generation. On the other hand, it was necessary to decrease the cytotoxicity of compound 25. From this generation the safest compound that showed the best antiviral activity was compound 13.

### Example 3

### Second-Generation Compounds

1. Chemistry. Second-Generation Compounds.

On the basis of the information generated by the first-generation inhibitors screened library, we concluded that urethane or benzofurane carbonyl groups appear to be preferable for the activity at the nitrogen firstly functionalized, as well as a urea group carrying electron-withdrawing groups at the other. As the next step compounds 27-41 with variation at R (electron-withdrawing groups) on the phenyl ring, and with tert-butoxyl or benzofuran-2-yl as R1, were synthesized following the same efficient and high yielded methodology by reaction of the compounds 6 and 9 with the appropriate isocyanate (Table 4).

TABLE 4
Second generation compounds

| | 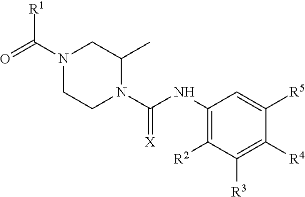 | | | | |
|---|---|---|---|---|---|
| Compound | R¹ | R² | R³ | R⁴ | R⁵ |
| 27 | O^{t}Bu | H | H | Cl | H |
| 28 | O^{t}Bu | H | H | CF₃ | H |
| 29 | O^{t}Bu | NO₂ | H | H | H |
| 30 | O^{t}Bu | Cl | H | H | CF₃ |
| 31 | O^{t}Bu | H | CF₃ | Cl | H |
| 32 | Benzofuran-2-yl | H | H | Cl | H |
| 33 | Benzofuran-2-yl | H | H | CF₃ | H |
| 34 | Benzofuran-2-yl | H | H | CN | H |
| 35 | Benzofuran-2-yl | H | H | F | H |
| 36 | Benzofuran-2-yl | NO₂ | H | H | H |
| 37 | Benzofuran-2-yl | F | H | H | H |
| 38 | Benzofuran-2-yl | Br | H | H | H |
| 39 | Benzofuran-2-yl | F | H | F | H |
| 40 | Benzofuran-2-yl | OCH₃ | H | H | H |
| 41 | Benzofuran-2-yl | H | H | H | H |

2. Biology. Second-Generation Compounds.

Compounds 27-41 were evaluated following the same assays described previously in order to determine their potential anti-HAdV activity.

| | Percentage | Percentage | |
|---|---|---|---|
| | of control | of plaque | |
| | HAdV5-GFP | formation | |
| Compound | infection^{a} | Inhibition^{b} | CC₅₀ (μM)^{c} |
| 27 | 36.97 ± 3.93 | 62.03 ± 0.26 | 150 ± 1.01 |
| 28 | 56.83 ± 6.23 | 30.65 ± 14.62 | 137.14 ± 24.33 |
| 29 | 0 | 50 ± 2.75 | — |
| 30 | 30.61 ± 7.96 | 96.25 ± 1.77 | 63.50 ± 8.22 |
| 31 | 71.52 ± 6.05 | 100 ± 0 | 20.80 ± 0.64 |
| 32 | 35.83 ± 5.13 | 6.69 ± 11.08 | 144.16 ± 20.75 |
| 33 | 60.01 ± 23.59 | 91.74 ± 4.52 | 82.16 ± 24.33 |
| 34 | 78.21 ± 8.65 | 83.16 ± 1.55 | 117.96 ± 30.17 |
| 35 | 0 | 92.42 ± 5.25 | 220.54 ± 28.99 |
| 36 | 0 | 91.45 ± 0.77 | 202.56 ± 4.09 |
| 37 | 0 | 50.92 ± 4.13 | — |
| 38 | 44.63 ± 12.29 | 55.79 ± 19.51 | — |
| 39 | 0 | 34.25 ± 13.56 | — |
| 40 | 0 | 16.44 ± 5.81 | — |
| 41 | 0 | 11.25 ± 26.52 | — |

Percentage inhibition of HAdV5-GFP in an ^{a}entry assay at 50 μM using the A549 cell line or in a ^{b}plaque assay at 10 μM using the 293β5 cell line.
^{c}Cytotoxic concentration 50.
The results represent means ± SD of triplicate samples from three independent experiments. See the Experimental Section for details

The effects on the activity of both the nature of the group and its position at the phenyl ring have been studied. As shown in Table 5, the 5 most active compounds obtained from this generation presented HAdV5-GFP plaque formation inhibition between 91 and 100% (compounds 30, 31, 33, 35 and 36); all of them with different electron-withdrawing groups at different positions of the phenyl ring.

For those tert-butoxycarbonyl derivatives 27-31, Cl, CF3 and ortho NO2 groups, did not improve the activity of compound 13 (27, 28 and 29 respectively, Table 5, entries 1-3). However, compounds 30 and 31 were very active, but very toxic as well (Table 5, entries 4 and 5). It is important to notice that compounds 30 and 31 possess a disubstituted phenyl ring (Cl and CF3). For those benzofuran-2-carbonyl derivatives, the lack of activity of compound 41, indicates that the presence of substituents on the phenyl ring is needed. Among the different groups evaluated at para position, Cl, CF3, CN and F (32, 33, 34 and 35 respectively), best activities were found with CF3 and F, but CF3 derivative 33 presented some cytoxicity.

For this generation the best data were obtained with compounds 35 and 36 (Table 5, entries 9 and 10), which were very active and with high CC50 (>200 □M). Compound 36 was synthesized in an attempt to obtain an analogue of 25 by changing the pattern of substitution of the phenyl ring from para (compound 25) to ortho position (compound 36), that resulted in a compound with similar activity and reduced toxicity. For other electron-withdrawing groups (F, Br) located at ortho position, no activity was detected (compounds 37 and 38).

### Example 4

### Third-Generation Compounds

1. Chemistry. Third-Generation Compounds.

A further round of optimization was performed to give a third generation of inhibitors by preserving structural features of the more active compounds from the second generation (tert-butoxyl or benzofuran-2-yl as R1 and a phenyl ring linked through a urea function with electron-withdrawing groups) and by changing the last point of structural variability of our general backbone, the substituent on the piperazine ring (FIG. 1). In this case, 2-phenylpiperazine was employed as starting material and through the general synthetic route shown in Scheme 3, monoacyl derivatives 43-45 were obtained in high yields.

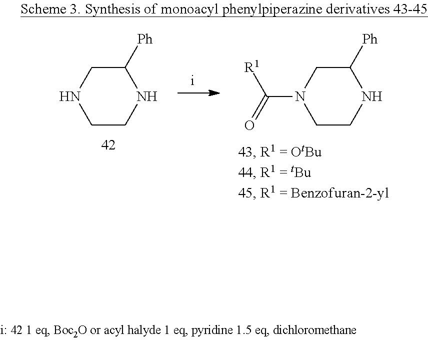

In the same way, the reaction of 43-45 with the appropriate isocyanate gave compounds 46-65 in high yields (Table 6).

TABLE 6
Third generation compounds

| | 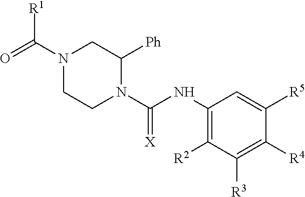 | | | | |
|---|---|---|---|---|---|
| Compound | R¹ | R² | R³ | R⁴ | R⁵ |
| 46 | O^{t}Bu | H | H | NO₂ | H |
| 47 | O^{t}Bu | H | H | Cl | H |
| 48 | O^{t}Bu | H | H | CF₃ | H |
| 49 | O^{t}Bu | H | H | F | H |
| 50 | O^{t}Bu | H | H | CN | H |
| 51 | O^{t}Bu | NO₂ | H | H | H |
| 52 | O^{t}Bu | Cl | H | H | CF₃ |
| 53 | O^{t}Bu | H | CF₃ | Cl | H |
| 54 | O^{t}Bu | H | H | OCH₃ | H |
| 55 | O^{t}Bu | H | H | CH₃ | H |
| 56 | ^{t}Bu | H | H | NO₂ | H |
| 57 | ^{t}Bu | H | H | CN | H |
| 58 | ^{t}Bu | H | H | OCH₃ | H |
| 59 | Benzofuran-2-yl | H | H | NO₂ | H |
| 60 | Benzofuran-2-yl | H | H | Cl | H |
| 61 | Benzofuran-2-yl | H | H | CF₃ | H |
| 62 | Benzofuran-2-yl | H | H | F | H |
| 63 | Benzofuran-2-yl | H | H | CN | H |
| 64 | Benzofuran-2-yl | NO₂ | H | H | H |
| 65 | Benzofuran-2-yl | Cl | H | H | CF₃ |

2. Biology. Third-Generation Compounds.

In the same way that all compounds previously prepared, the potential anti-HAdV activity of compounds 45-65 was evaluated (Table 7).

TABLE 7
Inhibition of HAdV infection in the entry and plaque assays and
effects on cellular viability for compounds 46-65.

| | Percentage | Percentage | |
|---|---|---|---|
| | of control | of plaque | |
| | HAdV5-GFP | formation | |
| Compound | infection^{a} | Inhibition^{b} | CC₅₀ (μM)^{c} |
| 46 | 80.15 ± 1.17 | 95.83 ± 4.03 | 161.32 ± 45.18 |
| 47 | 96.29 ± 1.13 | 86.27 ± 6.35 | 17.09 ± 4.24 |
| 48 | 74.54 ± 9.20 | 71.51 ± 0.11 | 42.13 ± 28.56 |
| 49 | 69.74 ± 2.23 | 75.84 ± 0.49 | 110.12 ± 8.09 |
| 50 | 45.12 ± 13.82 | 67.25 ± 16.54 | 91.28 ± 61.52 |
| 51 | 62.34 ± 10.12 | 100.00 ± 0 | 82.16 ± 9.06 |
| 52 | 89.17 ± 1.19 | 100.00 ± 0 | 93.36 ± 0.73 |
| 53 | 92.63 ± 0.25 | 100.00 ± 0 | 27.5 ± 0 |
| 54 | 39.30 ± 10.87 | 78.89 ± 1.57 | 133.28 ± 58.78 |
| 55 | 65.35 ± 23 | 63.02 ± 0.20 | 165.17 ± 17.38 |
| 56 | 60.28 ± 6.91 | 43.81 ± 13.24 | 102.36 ± 8.38 |
| 57 | 73.52 ± 10.20 | 83.97 ± 6.75 | 147.16 ± 37.99 |
| 58 | 0 | 70.09 ± 2.70 | 198.74 ± 33.58 |
| 59 | 83.89 ± 5.17 | 100 ± 0 | 193.9 ± 1.68 |
| 60 | 32.79 ± 3.13 | 100 ± 0 | 193.50 ± 9.19 |
| 61 | 60.79 ± 4.13 | 100 ± 0.00 | 70.69 ± 21.43 |
| 62 | 88.22 ± 1.14 | 98.81 ± 0.15 | 12.51 ± 0.79 |
| 63 | 62.51 ± 0.82 | 98.09 ± 0.33 | 199.84 ± 0.26 |
| 64 | 38.29 ± 8.44 | 100 ± 0 | 131.85 ± 6.01 |
| 65 | 29.84 ± 0.29 | 97.61 ± 1.12 | 130.8 ± 17.79 |

Percentage inhibition of HAdV5-GFP in an ^{a}entry assay at 50 μM using the A549 cell line or in a ^{b}plaque assay at 10 μM using the 293β5 cell line.
^{c}50% cell cytotoxic conbcentration.
The results represent means ± SD of triplicate samples from three independent experiments. See the Experimental Section for details.

In general terms, compounds from this generation presented high activity (only two out of 20 gave percentage of inhibition less than 70%). For those tert-butoxycarbonyl derivatives 46-55, only 46 presented high inhibitory activity (NO2 group) in absence of cytotoxicity, while Cl, CF3, F, and CN groups at para position did not improve activity. The presence of Cl or CF3 (47 and 48 respectively, Table 7, entries 2 and 3) resulted in significant cytotoxicity. In the same way, compounds 51, 52 and 53 presented high inhibitory activity, but exhibited significant cytotoxicity as well (Table 7, entries 6 and 7). It is important to notice that compound 51 possessed a NO2 grup at ortho position while 52, 53 possessed a disubstituted phenyl ring (Cl and CF3 at different positions). They were designed to be phenylpiperazine derivatives analogues of the corresponding methyl piperazine derivatives, 29, 30 and 31 respectively.

The replacement of the methyl by a phenyl gave the active compound 51 (29 was not active) but with certain cytotoxicity. In the case of compounds 52 and 53, the desired target was to get similar inhibitory activity than 30 and 31 but without their cytotoxicity Compound 52 resulted in a more active compound with less cytotoxicity (52 vs 30) whereas compound 53 resulted as cytotoxic as 31 despite presenting significant inhibitory activity. When a benzofuran-2-yl group is present those compounds possessing a para substituted phenyl ring were more active than their analogues tert-butoxycarbonyl derivatives. This fact gave not only the NO2 derivative as a new active compound 59, but also, Cl and CN ones (60 and 63). CF3 and F derivatives 61 and 62 were improved compounds in terms of anti-HAdV activity compared to 48 and 49, however they still exhibited high toxicity. The ortho NO2 derivative 64 was very active and non-cytotoxic, while its tert-butoxycarbonyl analogue 51 presented low toxicity. In addition the disubstituted benzofuran-2-yl derivative 65 was prepared as analogue of 52, presenting also high inhibitory activity and low cytotoxicity.

Finally due to the fact that the general backbone has been changed related to the previous generations, three phenylpiperazine derivatives (56-58) having an acyl group different from the two mentioned above, and a urea function were prepared and evaluated. The substituents located at the phenyl ring were both electron-withdrawing and electron-releasing groups. None of them gave anti-HAdV activity.

Compounds 46, 59, 60, 63, 64, and 65 were selected for their antiviral activity in the plaque assay, from 96 to 100% inhibition and their low cytotoxicity. These active compounds were further evaluated for measurement of 50% compound inhibition concentration (IC50), the selectivity index (SI) for each compound and also to gain some mechanistic understanding for inhibition (Table 8).

TABLE 8
2-Phenylpiperazines 46, 59, 60, 63, 64, and 65 IC50, CC50, and SI value.

| | IC₅₀ (μM)^{a} | | | Selectivity Index (SI)^{c} | | Virus yield reduction^{d} | |
|---|---|---|---|---|---|---|---|
| Compound | HAdV5 | HAdV16 | CC₅₀(μM)^{b} | HAdV5 | HAdV16 | HAdV5 | HAdV16 |
| 46 | 3.4 ± 0.96 | 4.8 ± 1.24 | 161.3 ± 45.18 | 47.3 | 33.6 | 211.7 ± 44.1 | 265.5 ± 29.1 |
| 59 | 2.1 ± 0.10 | 3.5 ± 1.03 | 193.9 ± 1.68 | 93.5 | 55.4 | 35.7 ± 13.5 | 34.6 ± 11.6 |
| 60 | 2.5 ± 1.17 | 3.4 ± 0.93 | 193.5 ± 9.19 | 79 | 56.9 | 9.9 ± 3.4 | 27.4 ± 16.9 |
| 63 | 4.7 ± 0.11 | 3.9 ± 1.12 | 199.8 ± 0.26 | 42.7 | 51.2 | 16.9 ± 5.2 | 60.9 ± 12.5 |
| 64 | 2.5 ± 0.03 | 2.3 ± 0.17 | 131.8 ± 6.01 | 53.3 | 57.3 | 34.5 ± 12.6 | 46.5 ± 10.8 |
| 65 | 1.1 ± 0.05 | 1.8 ± 0.2 | 130.8 ± 17.79 | 116.2 | 72.7 | 60.3 ± 15.2 | 33.2 ± 7.3 |

^{a}Inhibitory concentration 50.
^{b}Cytotoxic concentration 50.
^{c}Selectivity Index value was determined as the ratio of cytotoxic concentration 50 (CC50) to inhibitory concentration 50 (IC50) for each compound.
^{d}Fold-reduction in virus yield as the ratio of particles produced in the presence of DMSO divided by the yield in the presence of each one of the 2-phenylpiperazin derivatives (50 μM). The results represent means ± SD of triplicate samples from three independent experiments

2-Phenylpiperazines 46 and 59 reproducibly inhibited HAdV5 infection in a dose-dependent manner at high multiplicity of infection (MOI), 2,000 viral particles (vp)/cell. In subsequent screening using a lower input of virus (0.06 vp/cell), these compounds also showed dose-dependent activity with 96-100% inhibition of plaque formation at concentrations of 10 □M (Table 8). On the other hand, compounds 60, 63, 64 and 65 inhibited HAdV5 infection to a lesser extent in the entry assay while keeping the high dose-dependent inhibition in the plaque assay (Table 8). We also tested the antiviral activity of these 2- phenylpiperazines against a species B HAdV (HAdV16), and found similar levels of inhibition to those seen with HAdV5 in the plaque assay (Table 8). The IC50 values for the hit compounds against HAdV5 and HAdV16 are summarized in Table 8.

### Example 5

### Impact on HADV Entry

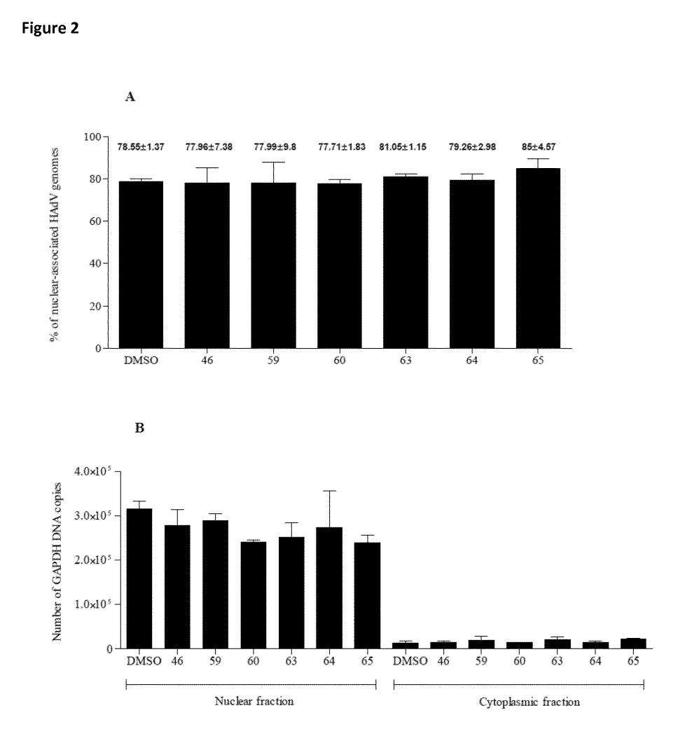

The entry assay we used for this work indicated that treatment with 2-phenylpiperazines 46, 59 and 63 inhibited expression of the HAdV-GFP transgene in a significant way at different levels depending on the compound, but this determination did not give us indications of their potential mechanism of inhibition. Only compounds 46 and 59 were able to reach an IC50 at concentrations below their CC₅₀ concentrations. To clarify if these molecules were able to block some of the steps of the entry pathway we carried out an assay to quantitatively measure the HAdV genome accessibility to the nucleus. After endosomal escape mediated by protein VI, the partially uncoated HAdV capsids are transported to the nuclear membrane via the microtubule network.25 At this point, the HAdV genome is imported into the nucleus along with protein VII, via the nuclear pore complex. We argued that if any of these 2-phenylpiperazines inhibited any of the steps of the HAdV entry that would be reflected in the number of HAdV genomes that reach the host nucleus after a synchronized infection. As showed in FIG. 2A, there were not significant differences in the amount of HAdV DNA isolated from the nucleus of cells treated with any of these 2-phenylpiperazines versus those treated with DMSO. We also measured the DNA copy number of the cellular housekeeping gene GAPDH in both the nucleus and the cytoplasm as a control for the purity of nuclear isolation. Our results indicated that we were specifically measuring the HAdV DNA that reached the nucleus and that the compounds did not alter any of the steps leading up to this late entry event (FIG. 2B).

### Example 6

### Impact on HADV Replication

The next step was to examine the effect that 2-phenylpiperazines 46, 59, 60, 63, 64, and 65 had on virus replication using a virus burst size assay which measures the production of infectious virus particles. We used the wild type virus and calculated the TCID50 of an infection in the presence and absence of the anti-HAdV molecules. As summarized in Table 8, treatment with our 2-phenylpiperazine derivatives was associated with overall reductions in virus yield of 9.9-211.7-fold for HAdV5 and 27.4-265.5-fold for HAdV16.

Additionally, we performed quantitative real-time PCR (qPCR) to measure HAdV DNA replication efficiency in the presence of these compounds. HAdV5-infected A549 cells were incubated for 24 h at 37° C. before washing to remove unbound virions. DNA was extracted at this early time point to avoid the influence of newly generated viral particles derived from subsequent rounds of infection occurring 32-36 hours post infection. The presence of compounds 46, 59, 60, 63, and 64 at 50 μM concentration significantly inhibited HAdV5 DNA replication by more than 50%, with no significant effect on the cellular control gene GAPDH (Table 9). Only compound 65 did not show a significant inhibition on HAdV5 DNA replication when compared to a control treated with the same concentration of DMSO.

TABLE 9
Effects of 2-phenylpiperazines 46, 59, 60, 63, 64 and
65 on HAdV5 and HCMV DNA replication.

| Compound | HadV5 | HCMV |
|---|---|---|
| 46 | 80.7 ± 2.5 | 99.6 ± 0.39 |
| 59 | 80.9 ± 0.2 | 99.9 ± 0.06 |
| 60 | 78.9 ± 4.2 | 98.2 ± 2.10 |
| 63 | 88.5 ± 2.7 | 99.9 ± 0.05 |
| 64 | 73.1 ± 13.3 | 97.9 ± 0.9 |
| 65 | 33.1 ± 0.5 | 98.2 ± 0.38 |

2-Phenyliperazines 46, 59, 60, 63, 64, and 65 inhibit DNA replication of HAdV5 and HCMV.

They reduced de novo production of HAdV5 and HCMV DNA copies compared to a positive control 24 h post-infection in a quantitative PCR assay (72 h in case of HCMV). The results represent means±SD of triplicate samples from three independent experiments.

### Example 7

### Impact on HCMV Replication

We explored the possible broad-spectrum inhibitory activity of these 2-phenylpiperazine derivatives on HCMV. Surprisingly, evaluation of HCMV DNA replication 72 h after infection of MRC-5 cells revealed significant inhibitions among those samples treated with our hit compounds and the control one treated with the same volume of DMSO (Table 9). Quantitative PCR for the GAPDH gene was included as control, again showing no differences between samples. These results suggest a mechanism for the inhibition of viral infection involving the machinery that participates in viral DNA replication.

### Example 8

Materials and Method for the New Families pf Anti-Bacterial Compounds

### 1. Strains

All of the strains used were Acinetobacter baumannii strains resistant to colistine: Resistance (R)≥4 Sensibility (S)≤2—All of these strains have been disclosed in Valencia R, Arroyo L A, Conde M, Aldana J M, Torres M J, Fernandez-Cuenca F, et al. Nosocomial outbreak of infection with pan-drug-resistant Acinetobacter baumannii in a tertiary care university hospital. Infection control and hospital epidemiology. 2009; 30(3):257-63. The specific strains used were the following: 1, 10, 11, 14, 16, 17, 19, 20, 21R, 22P, 24, 99 and 113.

### 2. Screening Procedure.

Evaluation of the inhibition of the bacterial growth of the 4 families of piperazines derivatives was performed by using a final concentration of 50 μM of each piperazine derivative in a concentration of 5×10⁵ CFU/ml of each strain of A. baumannii in 96-well plates.

These plates were incubated for 18 hours at 37° C.

### 3. Minimal Inhibitory Concentration) (MIC)

Microdilution in cell medium Mueller Hinton Broth II (MHBII). This technique was carried out solely in those piperazines which presented inhibitory activity in the screening study.

We used decreasing concentrations of each piperazine derivative (from 50 μM -0.05 μM) in a concentration of 5×10⁵ CFU/ml of each strain of A. baumannii in 96 well plates. These plates were incubated for 18 hours at a temperature 37° C.

### Example 9

### Results Screening (μM) First Family of Compounds

The compounds tested are illustrated in table 10 below.

TABLE 10

| Compuesto | R⁴ | R³ | R⁵ |
|---|---|---|---|
| Vip 499 | NO₂ | — | — |
| Vip 580 | Cl | — | — |
| Vip 584 | CN | — | — |
| Vip 585 | F | — | — |
| Vip 604 | CF₃ | — | — |
| Vip 590 | OCH₃ | — | — |
| Vip 591 | CH₃ | — | — |
| Vip 592 | — | CF₃ | CF₃ |

The results obtained are illustrated in table 11 below:

TABLE 11

| | Acinetobacter baumannii | | | | | | | | | | | | |
|---|---|---|---|---|---|---|---|---|---|---|---|---|---|
| Compuestos | Ab 1 | Ab 10 | Ab 11 | Ab 14 | Ab 16 | Ab 17 | Ab 19 | Ab 20 | Ab 21R | Ab 22P | Ab 24 | Ab 99 | Ab 113 |
| 499 (PROTOTIPO) | — | — | — | <50 | — | — | — | — | <50 | <50 | <50 | — | — |
| 580 | — | — | <50 | <50 | — | <50 | — | — | <50 | <50 | <50 | — | — |
| 584 | — | — | <50 | <50 | — | <50 | — | — | <50 | <50 | <50 | — | — |
| 585 | — | — | — | — | — | — | — | — | — | — | — | — | — |
| 604 | — | — | — | — | — | — | — | — | <50 | <50 | <50 | — | — |
| 590 | — | — | — | <50 | — | — | — | — | — | — | — | — | — |
| 591 | — | — | — | <50 | — | <50 | — | — | <50 | — | — | — | — |
| 592 | — | — | — | <50 | — | — | — | — | — | — | — | — | — |
| R-592 | — | — | — | — | — | — | — | — | <50 | — | — | — | — |
| S-592 | — | — | — | — | — | — | — | — | <50 | <50 | <50 | — | — |

Peptide concentration: 50 μM

Inoculum concentration: 5*105 UFC/ml

### Example 10

### Results CMI (μM) First Family of Compounds

The results obtained are illustrated in table 12 below

TABLE 12

| | Acinetabacter baumannii | | | | | |
|---|---|---|---|---|---|---|
| Compuestos | Ab 11 | Ab 14 | Ab 17 | Ab 21R | Ab 22P | Ab 24 |
| 499 (PROTOTIPO) | — | 25 | — | 50 | 25 | 25 |
| 580 | 50 | 50 | 50 | 50 | 50 | 50 |
| 584 | 50 | 50 | 25 | 50 | 50 | 50 |
| 585 | — | — | — | — | — | — |
| 604 | — | — | — | 25 | 25 | 25 |
| 590 | — | >50 | — | — | — | — |
| 591 | — | >50 | 50 | 50 | — | — |
| 592 | — | 3.12 | — | — | — | — |
| R-592 | — | — | — | 3.12 | — | — |
| S-592 | — | — | — | 3.12 | 12.5 | 3.12 |

Inoculum concentration: 5*105 UFC/ml

### Example 11

### Results Screening (μM) Second Family of Compounds

The compounds tested are illustrated in table 13 below, R corresponds to R₄:

TABLE 13

| Compuesto | R | R³ | R⁵ |
|---|---|---|---|
| Vip 611 | NO₂ | — | — |
| Vip 612 | Cl | — | — |
| Vip 613 | CN | — | — |
| Vip 616 | F | — | — |
| Vip 614 | CF₃ | — | — |
| Vip 621 | OCH₃ | — | — |
| Vip 619 | CH₃ | — | — |
| Vip 615 | — | CF₃ | CF₃ |

The results obtained are illustrated in table 14 below

TABLE 14

| | Acinetobacter baumannii | | | | | | | | | | | | |
|---|---|---|---|---|---|---|---|---|---|---|---|---|---|
| Compuestos | Ab 1 | Ab 10 | Ab 11 | Ab 14 | Ab 16 | Ab 17 | Ab 19 | Ab 20 | Ab 21R | Ab 22P | Ab 24 | Ab 99 | Ab 113 |
| 598 | — | — | — | — | — | — | — | — | <50 | <50 | <50 | — | — |
| 600 | — | — | — | — | — | — | — | — | <50 | <50 | <50 | — | — |
| 606 | — | — | — | — | — | — | — | — | <50 | — | — | — | — |
| 602 | — | — | — | — | — | — | — | — | <50 | — | — | — | — |
| 609 | — | — | — | <50 | — | — | — | — | <50 | <50 | <50 | — | — |
| 620 | — | — | — | <50 | — | — | — | — | <50 | — | — | — | — |
| 618 | — | — | — | <50 | — | — | — | — | <50 | <50 | — | — | — |
| 610 | — | — | — | <50 | — | — | — | — | <50 | <50 | <50 | — | — |
| R-610 | — | — | — | — | — | — | — | — | <50 | <50 | <50 | — | — |
| S-610 | — | — | — | — | — | — | — | — | <50 | <50 | <50 | — | — |

Peptide concentration: 50 μM Inoculum concentration: 5*105 UFC/ml

### Example 12

### Results CMI (μM) Second Family of Compounds

The results obtained are illustrated in table 15 below:

TABLE 15

Acinetobacter baumannii

| Compuestos | Ab 14 | Ab 21R | Ab 22P | Ab 24 |
|---|---|---|---|---|
| 598 | — | 50 | 50 | 50 |
| 600 | — | 50 | >50 | >50 |
| 606 | — | >50 | — | — |
| 602 | — | >50 | — | — |
| 609 | 50 | 25 | 25 | 25 |
| 620 | >50 | >50 | — | — |
| 618 | >50 | >50 | 50 | — |
| 610 | 50 | 6.25 | 6.25 | 6.25 |
| R-610 | — | 6.25 | 12.5 | 6.25 |
| S-610 | — | 6.25 | 6.25 | 12.5 |

Inoculum concentration: 5*105 UFC/ml.

### Example 13

### Results Screening (μM) Third Family of Compounds

The compounds tested are illustrated in table 16 below:

TABLE 16

| Compuesto | R | R³ | R⁵ |
|---|---|---|---|
| Vip 611 | NO₃ | — | — |
| Vip 612 | Cl | — | — |
| Vip 613 | CN | — | — |
| Vip 616 | F | — | — |
| Vip 614 | CF₃ | — | — |
| Vip 621 | OCH₃ | — | — |
| Vip 619 | CH₃ | — | — |
| Vip 615 | — | CF₃ | CF₃ |

The results obtained are illustrated in table 17 below:

TABLE 17

| | Acinetobacter baumannii | | | | | | | | | | | | |
|---|---|---|---|---|---|---|---|---|---|---|---|---|---|
| Compuestos | Ab 1 | Ab 10 | Ab 11 | Ab 14 | Ab 16 | Ab 17 | Ab 19 | Ab 20 | Ab 21R | Ab 22P | Ab 24 | Ab 99 | Ab 113 |
| 611 | — | — | — | — | — | — | — | — | <50 | <50 | — | — | — |
| 612 | — | — | — | <50 | — | — | — | — | <50 | <50 | <50 | — | — |
| 613 | — | — | — | <50 | — | — | — | — | <50 | <50 | <50 | — | — |
| 616 | — | — | — | — | — | — | — | — | <50 | <50 | — | — | — |
| 614 | — | — | — | <50 | — | — | — | — | <50 | <50 | <50 | — | — |
| 621 | — | — | — | <50 | — | — | — | — | <50 | <50 | — | — | — |
| 619 | — | — | — | <50 | — | — | — | — | <50 | <50 | — | — | — |
| 615 | — | — | — | <50 | — | — | — | — | <50 | <50 | <50 | — | — |
| R-615 | — | — | — | — | — | — | — | — | — | — | <50 | — | — |
| S-615 | — | — | — | — | — | — | — | — | — | — | — | — | — |

Peptide concentration: 50 μM Inoculum concentration: 5*105 UFC/ml

### Example 14

### Results CMI (μM) Third Family of Compounds

The results obtained are illustrated in table 18 below:

TABLE 18

Acinetobacter boumannii

| Compuestos | Ab 14 | Ab 21R | Ab 22P | Ab 24 |
|---|---|---|---|---|
| 611 | — | 25 | 12.5 | — |
| 612 | >50 | 12.5 | 12.5 | 25 |
| 613 | 25 | 25 | 50 | 25 |
| 616 | — | 50 | 25 | — |
| 614 | 12.5 | 25 | 6.25 | 3.12 |
| 621 | >50 | >50 | 50 | — |
| 619 | 50 | 50 | 25 | — |
| 619 | 50 | 3.12 | 1.56 | 25 |
| R-615 | — | — | — | 6.25 |
| S-615 | — | — | — | — |

Inoculum concentration: 5*105UFC/ml

### Example 15

### Results Screening (μM) Fourth Family of Compounds

The compounds tested are illustrated in table 19 below:

TABLE 19

| Compuesto | R | R³ | R⁵ |
|---|---|---|---|
| Vip 628 | NO₂ | — | — |
| Vip 629 | Cl | — | — |
| Vip 635 | CN | — | — |
| Vip 638 | F | — | — |
| Vip 633 | CF₃ | — | — |
| Vip 637 | OCH₃ | — | — |
| Vip 636 | CH₃ | — | — |
| Vip 634 | — | CF₃ | CF₃ |

The results obtained are illustrated in table 20 below

TABLE 20

| | Acinetobacter baumannii | | | | | | | | | | | | |
|---|---|---|---|---|---|---|---|---|---|---|---|---|---|
| Compuestos | Ab 1 | Ab 10 | Ab 11 | Ab 14 | Ab 16 | Ab 17 | Ab 19 | Ab 20 | Ab 21R | Ab 22P | Ab 24 | Ab 99 | Ab 113 |
| 628 | — | — | <50 | — | — | <50 | — | — | — | — | <50 | — | — |
| 629 | — | — | <50 | <50 | — | <50 | — | — | <50 | — | <50 | — | — |
| 635 | — | — | <50 | — | — | <50 | — | — | <50 | — | <50 | — | — |
| 638 | — | — | — | — | — | — | — | — | — | — | <50 | — | — |
| 633 | — | — | <50 | — | — | <50 | — | — | — | — | <50 | — | — |
| 637 | — | — | — | — | — | <50 | — | — | — | — | <50 | — | — |
| 636 | — | — | <50 | — | — | <50 | — | — | <50 | — | <50 | — | — |
| 634 | — | — | <50 | <50 | — | <50 | — | — | <50 | — | <50 | — | — |

Peptide concentration: 50 μM

Inoculum concentration: 5*105 UFC/ml

### Example 16

### Results CMI (μM) Fourth Family of Compounds

The results obtained are illustrated in table 21 below:

TABLE 21

| | Acinetobacter boumannii | | | | |
|---|---|---|---|---|---|
| Compuestos | Ab 11 | Ab 14 | Ab 17 | Ab 21R | Ab 24 |
| 628 | 50 | — | 25 | — | 50 |
| 629 | 25 | 25 | 25 | 50 | 50 |
| 635 | 50 | — | 50 | 50 | 50 |
| 638 | — | — | — | — | 50 |
| 633 | 25 | — | 25 | — | 50 |
| 637 | — | — | 50 | — | >50 |
| 636 | 50 | — | 25 | 25 | 50 |
| 634 | 6.25 | 3.12 | 3.12 | 3.12 | 6.25 |

Inoculum concentration: 5*105 UFC/ml

### Example 17

### Results Screening (μM) Fifth Family of Compounds and Further Results

The compounds tested are illustrated in table 22 below:

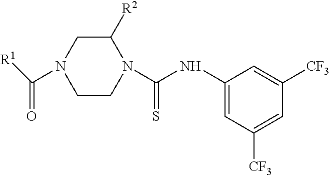

TABLE 22

| Compuesto | R¹ | R² |
|---|---|---|
| Vip 674 | CH₂^{t}But | Ph |
| Vip 679 | CH₂^{c}Hex | Ph |
| Vip 688 | CH₂Ph | Ph |
| Vip 695 | O^{t}But | Ph |
| Vip 692 | O^{t}But | H |

| | | | | YIELD | | |
|---|---|---|---|---|---|---|
| COMPOUNDS | Inhibition assay in plaque | IC50 plaque | Starting Inhibition assay | REDUCTION | PCR | CC50 |
| 572 | 30.37 ± 16.08 | | 58.12 ± 17.58 | | | |
| 574 | 22.78 ± 16.24 | | 13.96 ± 6.53 | | | |
| 580 | 73.43 ± 4.19 | | 64.11 ± 16.81 | | | 167.12 |
| 584 | 88.91 ± 15.92 | | 58.11 ± 25.72 | | | 175.00 |
| 585 | 92.91 ± 3.82 | | 75.54 ± 13.76 | 12.08 ± 2.78 | | 200.00 |
| 590 | 36.55 ± 30.13 | | 62.79 ± 17.03 | | | |
| 591 | 49.11 ± 4.73 | | 56.61 ± 17.69 | | | |
| 592 | 11.11 ± 19.25 | | 71.76 ± 15.00 | | | |
| 598 | 76.23 ± 22.02 | | 43.20 ± 8.65 | | | |
| 600 | 61.64 ± 25.29 | | 71.26 ± 15.15 | | | |
| 602 | 7.50 ± 15.00 | | 70.33 ± 15.41 | | | |
| 604 | 73.43 ± 22.49 | | 43.24 ± 17.18 | | | |
| 606 | 28.87 ± 31.82 | | 56.60 ± 17.69 | | | |
| 609 | 36.33 ± 32.43 | | 57.61 ± 17.62 | | | |
| 610 | 4.81 ± 9.34 | | 10.74 ± 14.03 | | | |
| 611 | 82.56 ± 15.63 | | 66.67 ± 16.31 | | | 148.10 |
| 612 | 89.33 ± 10.08 | | 72.26 ± 14.85 | | | 200.00 |
| 613 | 100.00 ± 0.00 | 1.98 | 81.77 ± 11.21 | 9.30 ± 2.90 | 0.00 ± 0.00 | 193.04 |
| 614 | 88.94 ± 10.32 | 1.88 | 54.09 ± 17.80 | | | 142.15 |
| 615 | 100.00 ± 0.00 | | 91.54 ± 5.83 | | | 82.40 |
| 616 | 100.00 ± 0.00 | 0.57 | 86.91 ± 8.60 | 30.53 ± 12.94 | 0.00 ± 0.00 | 143.36 |
| 618 | 76.83 ± 13.56 | | 71.61 ± 15.05 | | | |
| 619 | 95.79 ± 4.82 | 2.06 | 69.65 ± 15.59 | 39.05 ± 15.95 | 46.57 ± 14.79 | 122.21 |
| 620 | 58.11 ± 17.20 | | 32.52 ± 7.50 | | | |
| 621 | 25.53 ± 36.11 | | 11.53 ± 6.16 | | | |
| 628 | 98.21 ± 3.57 | 2.04 | 60.79 ± 17.31 | 25.59 ± 10.45 | 17.55 ± 30.80 | 210.38 |
| 629 | 84.56 ± 15.72 | | 59.51 ± 17.45 | | | 175.00 |
| 633 | 45.36 ± 16.56 | | 43.12 ± 17.16 | | | |
| 634 | 99.50 ± 1.58 | | 94.46 ± 3.87 | 33.42 ± 10.16 | 87.98 ± 11.78 | 129.74 |
| 635 | 98.36 ± 2.13 | | 60.06 ± 17.39 | 18.39 ± 5.44 | | 174.69 |
| 636 | 13.79 ± 25.70 | | 73.72 ± 14.39 | | | |
| 637 | 29.36 ± 36.56 | | 61.99 ± 17.15 | | | |
| 638 | 28.90 ± 23.21 | | 72.06 ± 14.91 | | | |
| 580 (1^{o} Family) | 73.43 ± 4.19 | | 64.11 ± 16.81 | | | 167.12 |
| 584 (1^{o} Family) | 88.91 ± 15.92 | | 58.11 ± 25.72 | | | 175.00 |
| 585 (1^{o} Family) | 92.91 ± 3.82 | | 75.54 ± 13.76 | 12.08 ± 2.78 | | 200.00 |
| 590 (1^{o} Family) | 36.55 ± 30.13 | | 62.79 ± 17.03 | | | |
| 591 (1^{o} Family) | 49.11 ± 4.73 | | 56.61 ± 17.69 | | | |
| 592 (1^{o} Family) | 11.11 ± 19.25 | | 71.76 ± 15.00 | | | |
| 604 (1^{o} Family) | 73.43 ± 22.49 | | 43.24 ± 17.18 | | | |
| 598 (2^{a} Family) | 76.23 ± 22.02 | | 43.20 ± 8.65 | | | |
| 600 (2^{a} Family) | 61.64 ± 25.29 | | 71.26 ± 15.15 | | | |
| 602 (2^{a} Family) | 7.50 ± 15.00 | | 70.33 ± 15.41 | | | |
| 606 (2^{a} Family) | 28.87 ± 31.82 | | 56.60 ± 17.69 | | | |
| 609 (2^{a} Family) | 36.33 ± 32.43 | | 57.61 ± 17.62 | | | |
| 610 (2^{a} Family) | 4.81 ± 9.34 | | 10.74 ± 14.03 | | | |
| 618 (2^{a} Family) | 76.83 ± 13.56 | | 71.61 ± 15.05 | | | |
| 620 (2^{a} Family) | 58.11 ± 17.20 | | 32.52 ± 7.50 | | | |
| 611 (3^{a} Family) | 82.56 ± 15.63 | | 66.67 ± 16.31 | | | 148.10 |
| 612 (3^{a} Family) | 89.33 ± 10.08 | | 72.26 ± 14.85 | | | 200.00 |
| 613 (3^{a} Family) | 100.00 ± 0.00 | 1.98 | 81.77 ± 11.21 | 9.30 ± 2.90 | 0.00 ± 0.00 | 193.04 |
| 614 (3^{a} Family) | 88.94 ± 10.32 | 1.88 | 54.09 ± 17.80 | | | 142.15 |
| 615 (3^{a} Family) | 100.00 ± 0.00 | | 91.54 ± 5.83 | | | 82.40 |
| 616 (3^{a} Family) | 100.00 ± 0.00 | 0.57 | 86.91 ± 8.60 | 30.53 ± 12.94 | 0.00 ± 0.00 | 143.36 |
| 619 (3^{a} Family) | 95.79 ± 4.82 | 2.06 | 69.65 ± 15.59 | 39.05 ± 15.95 | 46.57 ± 14.79 | 122.21 |
| 621 (3^{a} Family) | 25.53 ± 36.11 | | 11.53 ± 6.16 | | | |
| 628 (4^{a} Family) | 98.21 ± 3.57 | 2.04 | 60.79 ± 17.31 | 25.59 ± 10.45 | 17.55 ± 30.80 | 210.38 |
| 629 (4^{a} Family) | 84.56 ± 15.72 | | 59.51 ± 17.45 | | | 175.00 |
| 633 (4^{a} Family) | 45.36 ± 16.56 | | 43.12 ± 17.16 | | | |
| 634 (4^{a} Family) | 99.50 ± 1.58 | | 94.46 ± 3.87 | 33.42 ± 10.16 | 87.98 ± 11.78 | 129.74 |
| 635 (4^{a} Family) | 98.36 ± 2.13 | | 60.06 ± 17.39 | 18.39 ± 5.44 | | 174.69 |
| 636 (4^{a} Family) | 13.79 ± 25.70 | | 73.72 ± 14.39 | | | |
| 637 (4^{a} Family) | 29.36 ± 36.56 | | 61.99 ± 17.15 | | | |
| 638 (4^{a} Family) | 28.90 ± 23.21 | | 72.06 ± 14.91 | | | |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A composition comprising a compound having the following Formula (I): 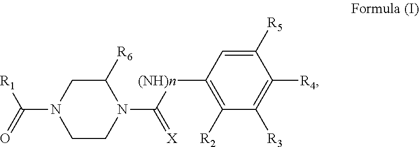
wherein:
R₁ is O^{t}Bu, ^{t}Bu, Ph, p-CH₃Ph, o-CH₃Ph, CH₂-^{t}Bu, CH₂-^{C}Hexyl, CH₂-Ph, CH═CHPh or Benzofuran-2-yl;
X is S or O;
R₂ is H, NO₂, Cl, F, Br or OCH₃;
R₃ is H or CF₃;
R₄ is H, NO₂, Cl, F, CH₃, CN, CF₃ or OCH₃;
R₅ is H or CF₃;
R₆ is H, CH₃ or Ph; and
n is 0 or 1.

2. The composition of claim 1, wherein the compound has the following Formula (V): 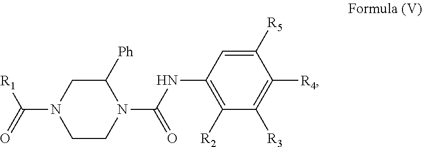
wherein:
R₁ is O^{t}Bu, ^{t}Bu, CH₂-^{t}Bu, Ph or Benzofuran-2-yl;
R₂ is H, NO₂, Cl, F, Br or OCH₃;
R₃ is H or CF₃;
R₄ is H, NO₂, Cl, F, CH₃, CN, CF₃ or OCH₃; and
R₅ is H or CF₃.

3. The composition of claim 1, wherein the compound has the following Formula (III): 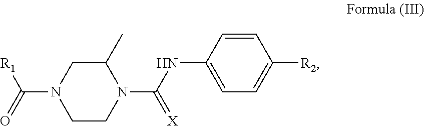
wherein:
R₁ is O^{t}Bu, ^{t}Bu, Ph or Benzofuran-2-yl;
R₂ is NO₂ or OCH₃; and
X is S or O.

4. The composition of claim 1, wherein the compound has the following Formula (IV): 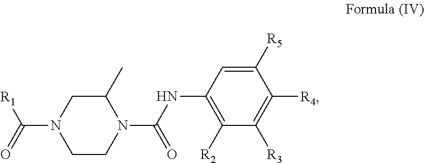
wherein:
R₁ is O^{t}Bu, ^{t}Bu, Ph or Benzofuran-2-yl;
R₂ is H, NO₂, Cl, F, Br or OCH₃;
R₃ is H or CF₃;
R₄ is H, NO₂, Cl, F, CH₃, CN, CF₃ or OCH₃; and
R₅ is H or CF₃.

5. The composition of claim 1, wherein the compound has the following Formula (II): 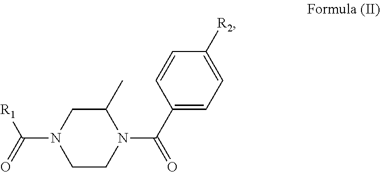
wherein:
R₁ is O^{t}Bu, ^{t}Bu, Ph or Benzofuran-2-yl; and
R₂ is NO₂ or OCH₃.

6. The composition of claim 2, wherein:
R₁ is O^{t}Bu or Benzofuran-2-yl;
R₂ is H, NO₂ or Cl;
R₃ is H;
R₄ is H, NO₂, Cl or CN; and
R₅ is H or CF₃.

7. The composition of claim 6, wherein the compound is selected from the group consisting of:
a. a compound wherein R₁ is O^{t}Bu; R₂ is H; R₃ is H; R₄ is NO₂; and R₅ is H;
b. a compound wherein R₁ is Benzofuran-2-yl; R₂ is H; R₃ is H; R₄ is NO₂; and R₅ is H;
c. a compound wherein R₁ is Benzofuran-2-yl; R₂ is H; R₃ is H; R₄ is Cl; and R₅ is H;
d. a compound wherein R₁ is Benzofuran-2-yl; R₂ is H; R₃ is H; R₄ is CN; and R₅ is H;
e. a compound wherein R₁ is Benzofuran-2-yl; R₂ is NO_{2;} R₃ is H; R₄ is H; and R₅ is H; and
f. a compound wherein R₁ is Benzofuran-2-yl; R₂ is Cl; R₃ is H; R₄ is H; and R₅ is CF₃.

8. The composition of claim 1, wherein the composition is a pharmaceutical composition and further comprises pharmaceutically acceptable excipients, carriers or diluents.

9. (canceled)

10. A method for treatment of an infection caused by a double-stranded DNA virus in a subject in need thereof, the method comprising administering a prophylactically or therapeutically effective amount of the composition of claim 1 to the subject.

11. The method of claim 10, wherein the double-stranded DNA virus is an adenovirus or herpesviruses.

12. The method of claim 11, wherein the herpesviruses is cytomegalovirus.

13. The method of claim 10, wherein the compound has the following Formula (V): 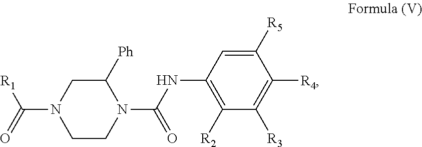
wherein:
R₁ is O^{t}Bu or Benzofuran-2-yl;
R₇ is H NO₂ or Cl;
R₃ is H;
R₄ is H, NO₂, Cl or CN; and
R₅ is H or CF₃.

14. The method of claim 13, wherein the compound is selected from the group consisting of:
a. a compound wherein R₁ is O^{t}Bu; R₂ is H; R₃ is H; R₄ is NO₂; and R₅ is H;
b. a compound wherein R₁ is Benzofuran-2-yl; R₂ is H; R₃ is H; R₄ is NO₂; and R₅ is H;
c. a compound wherein R₁ is Benzofuran-2-yl; R₂ is H; R₃ is H; R₄ is Cl; and R₅ is H;
d. a compound wherein R₁ is Benzofuran-2-yl; R₂ is H; R₃ is H; R₄ is CN; and R₅ is H;
e. a compound wherein R₁ is Benzofuran-2-yl; R₂ is NO₂; R₃ is H; R₄ is H; and R₅ is H.

15. The method of claim 10, wherein the subject is a human subject, and the double-stranded DNA virus is a human adenovirus.

16. The method of claim 15, wherein the subject is a human subject suffering from a respiratory disease, form conjunctivitis, gastroenteritis, HIV, or obesity; or the human subject is subjected to immunosuppressive therapies.

17. The method of claim 15, wherein the human adenovirus is selected from the group consisting of:
a. Species A and types 12, 18, 31;
b. Species B and types 3, 7, 11, 14, 16, 21, 34, 35, 50, 55;
c. Species C and types 1, 2, 5, 6, 57;
d. Species D and types 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54, 56;
e. Species E and type 4;
f. Species F and types 40 and 41; and
g. Species G and type 52.

18. The method of claim 17, wherein the human subject is suffering from a respiratory disease, form conjunctivitis, gastroenteritis, HIV, or obesity; or the human subject is subjected to immunosuppressive therapies.
